(19) United States
(12) Patent Application Publication
(10) Pub. No.: US 2005/0192259 A1
(43) Pub. Date: Sep. 1, 2005

(54) Aldosterone antagonist compositions for release during aldosterone acrophase

(75) Inventors: Susan Garthwaite, Evanston, IL (US); Leo K. Mathur, Vernon Hills, IL (US)

Correspondence Address: Pharmacia Corporation of Pfizer Inc;Corporate Patent Department, P.O. Box 1027, Chesterfield, MO, 63006, US

(73) Assignee: Pharmacia Corporation

(21) Appl. No.: 11/121,638

(22) Filed: May 4, 2005

Related U.S. Application Data
(63) Continuation of application No. 09/854,264, filed on May 11, 2001, now abandoned.
(60) Provisional application No. 60/203,637, filed on May 11, 2000.

(51) Int. Cl. A61K 31/56

(52) U.S. Cl. 514/169

(57) ABSTRACT

A pharmaceutical composition is provided for administration to a subject mammal such as a human exhibiting a diurnal cycle of plasma aldosterone concentration, the composition comprising a delayed-release formulation of an aldosterone antagonist drug, e.g., eplerenone, in a therapeutically effective amount. The delayed-release formulation, when administered about 6 hours to about 12 hours prior to the acrophase, results in a profile of plasma drug concentration that corresponds substantially to the diurnal cycle of plasma aldosterone concentration.

### CROSS REFERENCE TO RELATED PATENTS AND PATENT APPLICATIONS

This application is a continuation of U.S. patent application Ser. No. 09/854,264, filed May 11, 2001, which claims the priority benefit of U.S. provisional application Ser. No. 60/203,637, filed May 12, 2000, both of which are incorporated by reference herein in their entirety.

### FIELD OF THE INVENTION

The present invention relates to a pharmaceutical composition for treatment of circulatory disorders, including cardiovascular diseases such as hypertension, congestive heart failure and cardiomyopathy, and cardiovascular injury associated therewith. The invention relates more particularly to a pharmaceutical composition comprising as an active ingredient an aldosterone receptor antagonist, to a method of treatment comprising administering such a composition to a mammalian subject in need thereof, and to the use of such a composition in the manufacture of a medicament.

### BACKGROUND OF THE INVENTION

Aldosterone antagonists are known to be useful in treatment of hypertension and associated cardiac disease or insufficiency. The steroid drug spironolactone is an aldosterone antagonist that has been available, for example under the trademark Aldactone®, for many years for treatment of hypertension.

The compound methyl hydrogen 9,11α-epoxy-17α-hydroxy-3-oxopregn-4-ene-7α,21-dicarboxylate, γ-lactone (eplerenone) was first reported in U.S. Pat. No. 4,559,332 to Grob et al. that describes and claims a class of 9,11 -epoxy steroid compounds and their salts, together with processes for preparation of such compounds. These compounds are described as aldosterone antagonists that can be administered in a therapeutically effective amount to treat pathological conditions mediated by aldosterone such as hypertension, cardiac insufficiency and cirrhosis of the liver. U.S. Pat. No. 4,559,332 contains general references to formulations such as tablets and capsules for oral administration of these 9,11-epoxy steroid compounds including eplerenone.

International Patent Publication No. WO 98/25948 later disclosed additional synthetic processes for preparation of a class of 9,11 -epoxy steroid compounds and their salts, including eplerenone.

Eplerenone (formerly called epoxymexrenone) corresponds in structure to Formula I, below:

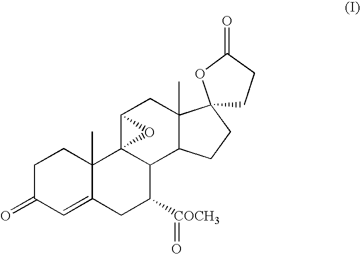

Spironolactone corresponds in structure to Formula II, below:

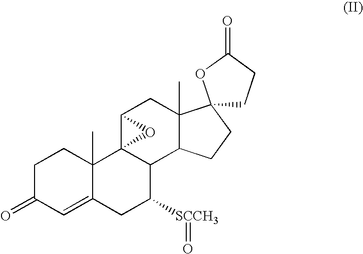

Spironolactone, however, exhibits antiandrogenic activity that can result in gynecomastia and impotence in men, and weak progestational activity that produces menstrual irregularities in women. Commercial medicaments of spironolactone contain 25, 50 or 100 mg doses of spironolactone in a formulation matrix comprising calcium sulfate dihydrate as a diluent, maize starch as a disintegrant, povidone K-30 as a binding agent, magnesium stearate as a lubricant, coating ingredients that include hydroxypropyl methylcellulose and polyethylene glycol 400, and flavoring and coloring agents. These commercial medicaments are designed for immediate release of spironolactone in the gastrointestinal tract of the recipient following oral administration.

Gasparo et al., J. Steroid Res., 22(1B):223-227 (1989) reported use of spironolactone and eplerenone in receptor binding studies. Spironolactone in a commercial formulation with a particle size of 5 μm and eplerenone at a particle size of 20 μm in a non-formulated composition were also used in vivo to study excretion of sodium in urine.

Several hormones have been shown to exhibit a circadian or diurnal rhythm of secretion in the human body. Aldosterone is one of those hormones whose secretion normally exhibits a diurnal cycle, having an acrophase, i.e., a period of maximal secretion, typically occurring in the late part of the sleep period and in the immediately following waking period, for example at about 0500 to about 0900 h (about 5 a.m. to about 9 a.m.) daily. See, for example, Cugini et al., Maturitas, 7:175-186 (1985); Kawasaki et al., Horm. Metab. Res., 22:636-639 (1990); Koopman et al., Neth. J. Med., 28(10):416-423(1985); Cugini et al., Chronobiologia, 12:155-165 (1985); Richards et al., Clinical Science, 73:489-495 (1987); and additional references cited therein.

Aldosterone acts on receptor molecules in many organs of the body. For example, by acting on receptor molecules in the tubules of the kidney, aldosterone promotes sodium retention, leading to increased water retention, which increases blood volume and blood pressure. As a consequence of the circadian rhythm of aldosterone secretion, there is a similar circadian rhythm of blood pressure with the greatest elevation of blood pressure typically occurring at about 6 a.m. to about 9 a.m., which also corresponds to an observed peak period for heart attacks to occur.

Aldosterone antagonist drugs such as spironolactone and eplerenone have not heretofore been formulated for administration at a significant time interval, i.e., several hours, prior to aldosterone acrophase, in such a way that the drug is released and maximally present only during the acrophase and is minimally present when aldosterone secretion is low.

Spironolactone has also been formulated in an immediate release medicament with the diuretic drug hydrochlorothiazide. Administration of that combination of drugs has not been suggested to be timed to correspond to the acrophase of aldosterone secretion, and therefore has been wasteful of the aldosterone antagonist portion of the medicament.

There is a need for compositions of aldosterone antagonist drugs such as eplerenone and spironolactone that release the drug at a time at which aldosterone secretion is highest so that maximal aldosterone antagonism can be achieved with minimal medicament. There is also a need for compositions that provide combination therapy of an aldosterone antagonist and another antihypertensive agent such that release or dissolution rates in the body correspond to aldosterone acrophase for the aldosterone antagonist and to a different time for the other antihypertensive agent. The present invention provides solutions to both of those needs.

### SUMMARY OF THE INVENTION

The present invention relates to an orally deliverable delayed-release formulation of an aldosterone antagonist and to a treatment method using the same, the formulation being designed to release the aldosterone antagonist in the gastrointestinal tract of a mammal, preferably a human, recipient at a time corresponding to the natural acrophase of aldosterone secretion by the recipient, so that the aldosterone antagonist is present in the bloodstream at its maximal amount at about the same time that aldosterone secretion is at its maximum.

Thus, the invention provides a pharmaceutical composition for administration to a subject mammal exhibiting a diurnal cycle of plasma aldosterone concentration having an acrophase, the composition comprising a delayed-release formulation of an aldosterone antagonist drug, preferably eplerenone, in a therapeutically effective amount. The delayed-release formulation is such that, when the composition is orally administered about 6 to about 12 hours prior to the acrophase, a profile of plasma drug concentration corresponding substantially to the diurnal cycle of aldosterone concentration, for example substantially as depicted in FIG. 1 herein, is exhibited.

Normally a lag of about 1 to about 4 hours, typically about 2 hours, is observed from the time an immediate-release formulation of an aldosterone antagonist drug such as eplerenone is orally administered to the time the drug reaches a therapeutic level in blood serum. Accordingly for the delayed-release formulation of the present invention it is contemplated that substantial release of the drug in the gastrointestinal tract begins about 2 hours before aldosterone acrophase, to provide a profile of blood serum concentration substantially as depicted in FIG. 1.

Thus, in one aspect, the invention contemplates a composition comprising a delayed-release formulation of an aldosterone antagonist drug, preferably eplerenone, in a therapeutically effective amount which exhibits a release profile, as determined by a suitable test, in which:
(a) zero to about 20%, and preferably zero to about 10%, by weight of the drug is released from the formulation at about 4 hours after initiation of the test; and
(b) about 50% to 100%, and preferably about 70% to 100%, by weight of the drug is released from the formulation within a time period of about 3 hours beginning at a time t that is about 4 to about 12 hours, preferably about 5 to about 10 hours, after initiation of the test.

An example of a suitable test is an in vitro dissolution test conducted according to U.S. Pharmacopeia 24 (2000), Test No. 711, using apparatus 2 (paddle) at 50 rpm, with an aqueous dissolution medium containing 1% sodium dodecyl sulfate (SDS) at 37° C. In this test dissolution in the aqueous medium provides the measure of release as specified above.

A “therapeutically effective amount” as specified above is an amount of the aldosterone antagonist that upon release in the gastrointestinal tract and subsequent absorption into the body of a recipient mammal, preferably a human, provides any therapeutic or prophylactic benefit. In a particular aspect of the invention, the “therapeutically effective amount” of aldosterone antagonist in a delayed-release formulation is an amount sufficient to lower blood pressure during aldosterone acrophase in the recipient mammal.

Another aspect of the invention contemplates a composition that further comprises a second formulation. The second formulation comprises a therapeutically effective amount, preferably an amount sufficient to lower blood pressure of a recipient mammal, of an antihypertensive agent. The antihypertensive agent in the second formulation can be an aldosterone antagonist, and can even be the same aldosterone antagonist, e.g., eplerenone, that is present in the delayed-release formulation, but is preferably other than an aldosterone antagonist. The second formulation preferably exhibits a release profile that is different from that of the delayed-release formulation; in particular it preferably releases its antihypertensive agent predominantly at a time or times other than immediately prior to or during aldosterone acrophase, when the aldosterone antagonist is being released from the delayed-release formulation. Accordingly in this preferred embodiment the second formulation can be an immediate-release formulation, a sustained-release formulation or a delayed-release formulation timed to release its antihypertensive agent substantially before or after aldosterone acrophase. The second antihypertensive agent is preferably selected from the group consisting of a diuretic, a sympatholytic agent, an angiotensin-converting enzyme (ACE) inhibitor, a calcium channel blocker, a direct vasodilator, a renin inhibitor, and an angiotensin II antagonist.

A method of treating a mammal exhibiting (a) circadian rhythm in aldosterone secretion having an acrophase and (b) an aldosterone-mediated disease or disorder such as elevated blood pressure is also contemplated. This method comprises administering to the mammal such as a human or veterinary animal, e.g., a companion, farm or exotic animal, a composition of the invention as described above, preferably such a composition wherein the aldosterone antagonist in the delayed-release formulation is eplerenone.

According to this method of the invention, the composition is administered orally about 6 to about 12 hours before the acrophase of aldosterone secretion. In a related embodiment, where the mammal is a human exhibiting aldosterone acrophase at the end of a sleep period and an immediately following part of a waking period, the composition is administered orally prior to the sleep period, for example at bedtime.

The present invention has several benefits and advantages.

One benefit of the invention is that by timing release of the aldosterone antagonist drug to correspond to the acrophase of aldosterone secretion as described herein, a lesser amount of the drug can be used, thereby lessening the possibilities for undesired side effects.

Another benefit of the invention is that by timing release of the aldosterone antagonist to correspond to the acrophase of aldosterone secretion as described herein, the rise in blood pressure that accompanies that secretion can be reduced with concomitant benefits in other aspects of cardiovascular health.

Another benefit of the invention is that use of two antihypertensive agents, one of which is an aldosterone antagonist timed for release to correspond to aldosterone acrophase as described herein, can provide a more encompassing treatment of high blood pressure and related cardiovascular problems, while minimizing the amount of active agents used. In addition, use of a combination of antihypertensive agents having different mechanisms can provide a better outcome in some situations than use of either antihypertensive agent alone.

Another benefit of the invention is the provision of a dosage form that can be administered at a convenient time of the day, e.g., before going to bed, and thereby improve compliance and avoid necessity for awakening the recipient during a sleep period. Pre-sleep administration can also reduce nocturnal diuresis.

Still further benefits and advantages of the invention will become apparent to the skilled worker from the disclosure that follows.

### BRIEF DESCRIPTION OF THE DRAWING

FIG. 1 shows a schematic representation of a diurnal cycle of aldosterone concentration 10 in blood plasma of a subject, the diurnal cycle exhibiting a period of acrophase 21 that occurs at the end of a sleep period 20. The shaded area 12 represents a zone in which plasma concentration of an aldosterone antagonist drug, e.g., eplerenone, is found when a composition of the invention is administered once daily during a pre-sleep period 22. The bold line 11 represents one example of the course of blood plasma concentration of the drug over a single day, when administered in accordance with the invention.

### DETAILED DESCRIPTION OF THE INVENTION

Aldosterone antagonists such as spironolactone and eplerenone have been found useful as antihypertensive agents. These agents are understood to interact with an aldosterone receptor in the kidneys resulting in a mild natriuresis and decreased potassium and hydrogen ion excretion.

These agents therefore exhibit their activity in the presence of aldosterone, secretion of which, as described above, typically follows a diurnal or circadian rhythm in humans, with a maximal amount (acrophase) being present in the blood at about 5 a.m. to about 9 a.m., and a minimal amount being present about twelve hours earlier. The exact time and duration of acrophase varies according to the individual subject and according to the subject's pattern of activity and sleep, among other factors. The acrophase of aldosterone secretion is coincident with a rise in blood pressure and is also a time at which heart attacks frequently occur.

Commercially available spironolactone-containing formulations are typically administered several times (e.g., 2-4 times) each day as immediate-release formulations. Such formulations are useful and are effective in lowering the recipient's blood pressure. However, such spironolactone-containing formulations can be wasteful of the active ingredient because of the relatively low concentration of aldosterone in the blood for most of the part of the day in which the recipient is awake. Spironolactone is rapidly metabolized, and like most pharmaceuticals, can have undesired side effects.

Extended-release, otherwise known as sustained-release, formulations of an antihypertensive formulation of eplerenone are disclosed in a co-assigned International Patent Application No. PCT/US 99/29136. However, the formulations disclosed therein typically release eplerenone over a period of up to about six hours after administration, and are not disclosed to be administered in such a way as to provide maximal release of the active agent immediately prior to or during aldosterone acrophase.

For example, a composition described in PCT/US 99/29136 as a “six hour CR tablet” has a 50% in vitro dissolution time of 6 hours, i.e., 50% of the eplerenone dissolves in the first 6 hours of the dissolution test. In an in vivo study in humans, plasma concentration of eplerenone peaked 4 hours after oral administration.

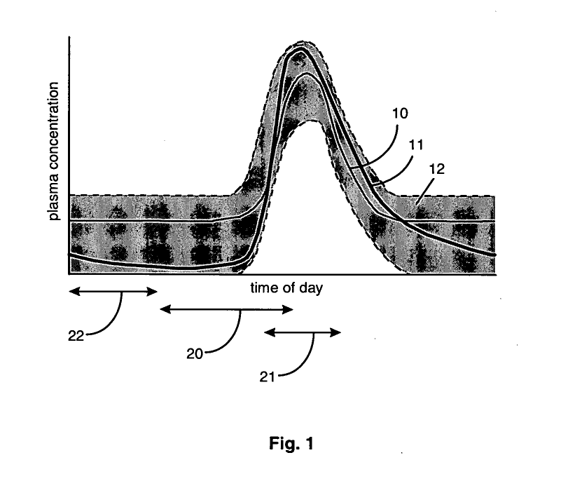

The present invention provides a pharmaceutical composition for administration to a subject mammal exhibiting a diurnal cycle of aldosterone concentration in blood serum having an acrophase, the composition comprising a delayed-release formulation of an aldosterone antagonist drug, preferably eplerenone, in a therapeutically effective amount. The delayed-release formulation is such that, when the composition is orally administered about 6 to about 12 hours prior to the acrophase, a profile of blood serum concentration of the drug corresponding substantially to the diurnal cycle of aldosterone concentration, substantially as depicted in FIG. 1 herein, is exhibited.

Referring to FIG. 1, a profile of blood serum (plasma) concentration of an aldosterone antagonist drug that falls in the shaded area 12 is provided by a composition of the invention if administered during the period 22 prior to a sleep period 20. This profile substantially matches the diurnal cycle of aldosterone concentration in plasma, represented by the line 10. In particular, during aldosterone acrophase 21, drug concentration in the plasma is much higher than at other times of the day when, because of the low level of aldosterone secretion, little benefit would be obtained from the drug.

Illustratively, a profile of eplerenone concentration provided by a contemplated delayed-release formulation is shown as the bold line 11. In this example, following oral administration during the pre-sleep period 22, very little release of the drug occurs for at least about 4 hours. At about the time aldosterone levels in the plasma begin to rise, in this example about 6 hours after the end of the pre-sleep period 22, the concentration of eplerenone in the plasma also begins to rise, reflecting an onset of release of the drug in the gastrointestinal tract, typically about 2 hours earlier. In this example release occurs over a very short period so that a very rapid rise in eplerenone concentration in the plasma is observed, followed by a gradual fall in eplerenone concentration as the drug is cleared from the bloodstream.

In one embodiment, a composition of the invention comprises a delayed-release formulation of an aldosterone antagonist drug, preferably eplerenone, in a therapeutically effective amount which exhibits a release profile, as determined by a suitable test, in which:
(a) zero to about 20%, and preferably zero to about 10%, by weight of the drug is released from the formulation at about 4 hours after initiation of the test; and
(b) about 50% to 100%, and preferably about 70% to 100%, by weight of the drug is released from the formulation within a time period of about 3 hours beginning at a time t that is about 4 to about 12 hours, preferably about 5 to about 10 hours, after initiation of the test.
   A preferred test is an in vitro dissolution test conducted according to U.S. Pharmacopeia 24 (2000), Test No. 711, using apparatus 2 (paddle) at 50 rpm, with an aqueous dissolution medium containing 1% sodium dodecyl sulfate (SDS) at 37° C. Unless otherwise indicated, the phrase “dissolution test”, “dissolution assay” or “dissolution study” herein refers to this USP test.

The subject mammal is preferably a human. Besides being useful for human treatment, a contemplated composition is also useful for veterinary treatment of companion animals, exotic animals and farm animals, including rodents and the like. More preferred non-human animals include horses, dogs and cats.

A contemplated composition contains the aldosterone antagonist at least in a delayed-release formulation. Thus, when only an aldosterone antagonist is present, the antagonist is in a delayed-release formulation. When a second antihypertensive agent is also present, that second agent can be present as an immediate-release formulation, an extended-release formulation, or a second delayed-release formulation. When the second antihypertensive agent is the same or another aldosterone antagonist, the formulation containing it is an immediate-release or extended-release formulation.

An immediate-release formulation of a drug is generally designed to release the drug immediately upon ingestion. In such formulations, greater than about 90% of the drug is dissolved within about 0.5 hours of initiation of the designated in vitro dissolution study.

An extended-release formulation releases the active ingredient over time and typically permits at least a two-fold reduction in dosing frequency as compared to a formulation that releases the active agent substantially immediately upon ingestion. Extended-release formulations are also known in the art as sustained-release formulations. An extended-release formulation contemplated here releases some of the second antihypertensive agent relatively quickly, with at least about 50% of the agent being dissolved at a stated time such as 2, 4 or 6 hours after initiation of the dissolution assay.

A delayed-release formulation of a drug releases the drug at any time other than promptly after administration. Enteric-coated tablets or pills are examples of delayed-release formulations. As is well known, an enteric coating permits a formulation to transit the stomach without dispensing the drug until a time when the formulation has reached the intestines. At that time release may begin immediately or be subject to a further delay until the enteric coating has sufficiently eroded, dissolved or dispersed in the intestinal fluid.

A delayed-release formulation contemplated here releases a minimal amount of eplerenone or other aldosterone antagonist in the first several hours (e.g., about 4 to about 12 hours) after ingestion. For example, zero to about 20%, and preferably zero to about 10%, of the eplerenone is dissolved at about 4 hours in the designated in vitro dissolution assay. Preferably substantially no eplerenone is released at about 4 hours in this assay.

The length of the delay period, during which release of the aldosterone antagonist drug is minimal, determines an appropriate time of day for administration of the delayed-release formulation. For example, if acrophase begins around 6 a.m., release of the drug should begin about 1-2 hours earlier, say around 4 a.m. to 5 a.m., to ensure a high level of the drug in plasma at the time aldosterone secretion is maximal. Thus a formulation providing 4-5 hours delayed release is suitable for administration around midnight, whereas one providing 11-12 hours delayed release should be administered around 5 p.m. A preferred time of administration is close to bedtime; thus for a subject who normally goes to bed at about 10 p.m. and who exhibits aldosterone acrophase around 6 a.m., a formulation providing about 6-7 hours delayed release is ideal. One of skill in the art will readily derive from information provided herein an appropriate delayed-release period for any particular situation.

The time, herein designated “t”, that marks the end of the delay period and commencement of substantial release of the aldosterone antagonist is thus, in the designated dissolution test, about 4 to about 12 hours after initiation of dissolution. Within a period of about 3 hours following time t, about 50% to 100%, and preferably about 70% to 100%, of the aldosterone antagonist drug is dissolved. The formulation thus releases the majority of the drug relatively quickly, e.g., within 1-3 hours. This is similar to the release profile of an immediate-release formulation, except that substantial dissolution begins at a time referred to herein as “t”, about 4 to about 12 hours after initiation of dissolution in the designated test.

A contemplated composition is intended for once per day administration. As such, the composition provides a therapeutically effective amount, for example an antihypertensive amount, of the aldosterone antagonist at a time that is substantially coincident with acrophase of aldosterone secretion. By comparison with immediate-release and extended-release formulations, a delayed-release formulation of the invention results in a diminished presence of the drug during the period of several hours following administration. The drug is unneeded during that period because the concentration of aldosterone is relatively low at that time. By reducing or eliminating unneeded drug through the delayed-release formulation of the invention, a substantially reduced dosage of aldosterone antagonist is possible. Exemplary reductions in daily dose are at least about 25%, and preferably at least about 50%.

Reduction or elimination of excess active agent not only avoids wastefulness but also minimizes undesirable side effects. Examples of possible undesirable side effects that can be minimized by use of compositions of the invention include, but are not limited to, gastrointestinal irritation, and antiandrogenic and progestational activity. Reducing the presence of the aldosterone antagonist drugs at times other than aldosterone acrophase can also reduce the incidence of hyperkalemia that is sometimes associated with such drugs.

A contemplated composition is advantageously used to block aldosterone receptors and, among other pharmacological actions, can increase sodium and water excretion with a concomitant potassium-sparing effect. Such compositions can be specifically employed for the prophylaxis and treatment of cardiovascular diseases such as heart failure; hypertension (especially the management of mild to moderate hypertension); edema associated with liver insufficiency; post-myocardial infarction; cirrhosis of the liver; stroke prevention; and reduction of heart rate for subjects exhibiting an accelerated heart rate.

Compositions of the present invention are useful for administration of 9,11-epoxy-20-spiroxane compounds other than eplerenone, particularly those 9,11-epoxy-20-spiroxane compounds that are aldosterone antagonists. These compositions can be prepared as set forth in this application by replacing eplerenone with a comparable weight fraction of the desired 9,11-epoxy-20-spiroxane. The 9,11-epoxy-20-spiroxane compounds used in the preparation of such compositions can be prepared, for example, as set forth in above-cited U.S. Pat. No. 4,559,332. Such 9,11-epoxy-spiroxanes include, but are not limited to, the following compounds:
9α,11α-epoxy-7α-methoxycarbonyl-15β,16β-methylene-20-spirox-4-ene-3,21-dione;
9α,11α-epoxy-7α-isopropoxycarbonyl-20-spirox-4-ene-3,21-dione;
9α,11α-epoxy-7α-ethoxycarbonyl-20-spirox-4-ene-3,21-dione;
9α,11α-epoxy-6β,7β-methylene-20-spirox-4-ene-3,21-dione;
9α,11α-epoxy-6β,7β; 15β,16β-bis-methylene-20-spirox-4-ene-3,21-dione;
9α,11α-epoxy-17β-hydroxy-6β,7β-methylene-3-oxo-17α-pregn-4-ene-21-carboxylic acid;
9α,11α-epoxy-17β-hydroxy-6β,7β-methylene-3-oxo-17α-pregn-4-ene-21-carboxylic acid methyl ester;
9α,11α-epoxy-17β-hydroxy-6β,7β;15β,16β-bis-methylene-3-oxo-17α-pregn-4-ene-21-carboxylic acid methyl ester;
9α,11α-epoxy-6β,7β-methylene-20-spiroxa-1,4-diene-3,21-dione;
9α,11α-epoxy-17β-hydroxy-7α-methoxycarbonyl-3-oxo-17α-pregn-4-ene-21-carboxylic acid;
9α,11α-epoxy-17β-hydroxy-3-oxo-17β-pregn-4-ene-7α,21-dicarboxylic acid dimethyl ester;
9α,11α-epoxy-17β-hydroxy-7α-isopropoxycarbonyl-3-oxo-17α-pregn-4-ene-21-carboxylic acid;
9α,11α-epoxy-17β-hydroxy-7α-ethoxycarbonyl-3-oxo-17α-pregn-4-ene-21-carboxylic acid;
9α,11α-epoxy-6α,7α-methylene-20-spirox-4-ene-3,21-dione;
9α,11α-epoxy-17β-hydroxy-3-oxo-17α-pregn-4-ene-7α,21-dicarboxylic acid dimethyl ester; and
9α,11α-epoxy-17β-hydroxy-15β,16β-methylene-3-oxo-17α-pregn-4-ene-7α,21-dicarboxylic acid dimethyl ester;
and pharmaceutically acceptable salts thereof.

Treatment of Specific Conditions and Disorders

For treatment of heart failure, an eplerenone composition of the invention is preferably administered at a daily dosage of about 10 mg to about 150 mg, more preferably about 15 mg to about 100 mg, for example about 25 mg. A daily dose of about 0.15 to about 2.1 mg/kg body weight (based upon an average body weight of about 75 kg), preferably about 0.15 to about 0.75 mg/kg body weight, for example about 0.5 mg/kg body weight, can be appropriate. The daily dose is preferably administered in a once-per-day dosage regimen, about 6 to about 12 hours prior to aldosterone acrophase.

For treatment of hypertension, an eplerenone composition of the invention is preferably administered at a daily dosage of about 30 mg to about 250 mg, more preferably about 30 mg to about 125 mg, for example about 75 mg. A daily dose of about 0.5 to about 3.0 mg/kg body weight, preferably about 0.5 to about 1.5 mg/kg body weight, for example about 1.0 mg/kg body weight, can be appropriate. The daily dose is preferably administered in a once-per-day dosage regimen, about 6 to about 12 hours prior to aldosterone acrophase.

For treatment of edema associated with liver insufficiency, an eplerenone composition of the invention is preferably administered at a daily dosage of about 25 mg to about 400 mg, more preferably about 75 mg to about 300 mg, for example about 100 mg to about 200 mg. A daily dose of about 0.5 to about 5.5 mg/kg body weight, preferably about 0.6 to about 4.0 mg/kg body weight, for example about 3.0 mg/kg body weight, can be appropriate. The daily dose is preferably administered in a once-per-day dosage regimen, about 6 to about 12 hours prior to aldosterone acrophase.

In general, a composition of the invention provides a daily dosage of aldosterone antagonist such as eplerenone sufficient to cause an increase, typically of at least 10%, in blood serum renin concentration and an increase, typically of at least about 50%, in blood serum aldosterone concentration in a human recipient over an interval of about 24 hours after oral administration.

A contemplated composition provides a daily dosage of an aldosterone antagonist such as eplerenone sufficient to cause an increase in urinary log₁₀(sodium/potassium ratio) in a human recipient over an interval of about 24 hours after oral administration.

A contemplated composition provides a daily dosage of an aldosterone antagonist such as eplerenone sufficient to cause an average decrease of at least about 5% in diastolic blood pressure in humans over an interval of about 24 hours after oral administration.

Combination Composition

Another embodiment of the invention contemplates a composition that comprises a formulation as described above along with a second formulation that contains a therapeutically effective amount, for example a blood pressure lowering amount, of a second antihypertensive agent. In one aspect, the second antihypertensive agent is an aldosterone antagonist and can be the same as the first named active agent; e.g., eplerenone or spironolactone. In another aspect, the second antihypertensive agent is other than an aldosterone antagonist. Whether the same or preferably a different active agent, the second antihypertensive agent is present in a second formulation that is different from the delayed-release formulation containing an aldosterone antagonist.

Exemplary second antihypertensive agents include a diuretic, a sympatholytic agent, an ACE inhibitor (including a vasopeptidase), a calcium channel blocker, a β-adrenergic blocking agent, an α-adrenergic blocking agent, a mixed β-adrenergic and α-adrenergic blocking agent, a ganglion blocking agent; a peripherally acting sympatholytic agent, a direct vasodilator, a renin inhibitor, and an angiotensin II antagonist. Illustrative compounds in the above categories are listed in the table below:

| | Adult maintenance |
|---|---|
| Compound | dose (mg/day) |
| Diuretics: | |
| Benzothiadiazine-type diuretics: | |
| Thiazides: | |
| Chlorothiazide | 250-1000 |
| Hydrochlorothiazide | 12.5-100 |
| Bendroflumethiazide | 5-20 |
| Benzthiazide | 25-100 |
| Hydroflumethiazide | 25-100 |
| Methychlothiazide | 2.5-5 |
| Polythiazide | 2-4 |
| Trichlormethiazide | 2-4 |
| Indapamide | 1.25-5 |
| Phthalimidines: | |
| Chlorthalidone | 12.5-100 |
| Metolazone | 0.5-5 |
| Quinazolines: | |
| Quinethazone | 50-100 |
| Loop diuretics: | |
| Furosemide | 20-1000 |
| Ethacrynic acid | 50-400 |
| Bumetanide | 0.5-2 |
| Potassium-sparing diuretics: | |
| Spironolactone | 50-100 |
| Eplerenone | |
| Triamterene | 50-100 |
| Amiloride | 5-10 |
| Sympatholytic agents: | |
| Centrally acting agents: | |
| Methyldopa | 500-2000 |
| Clonidine | 0.2-0.8 |
| Clonidine patch | 0.1-0.3 |
| Guanfacine | 1-3 |
| Guanabenz | 8-64 |
| Reserpine and rauwolfia alkaloids | 0.1-0.25 |
| β-Adrenergic blocking agents: | |
| Propranolol | 40-640 |
| Propranolol sustained release | 80-640 |
| Carteolol | 2.5-10 |
| Betaxolol | 10-20 |
| Metoprolol | 100-450 |
| Metoprolol sustained release | 50-400 |
| Bisoprolol | 2.5-40 |
| Atenolol | 50-100 |
| Nadolol | 40-320 |
| Timolol | 20-60 |
| Pindolol | 10-60 |
| Acebutolol | 400-1200 |
| Penbutolol | 20 |
| α-Adrenergic blocking agents: | |
| Prazosin | 2.5-20 |
| Terazosin | 1-20 |
| Doxazosin | 2-16 |
| Mixed α- and | |
| β-adrenergic blocking agents: | |
| Labetalol | 200-1200 |
| Ganglion blocking agents: | |
| Mecamylamine | 2.5-25 |
| Peripherally acting | |
| sympatholytic agents: | |
| Guanethidine | 10-50 |
| Angiotensin-converting enzyme | |
| (ACE) inhibitors: | |
| Captopril | 75-450 |
| Enalapril | 5-40 |
| Lisinopril | 10-40 |
| Quinapril | 5-80 |
| Ramipril | 2.5-20 |
| Benazepril | 10-80 |
| Fosinopril | 10-80 |
| Perindopril | 2-16 |
| Vasopeptidase inhibitors: | |
| Omapatrilat | |
| Calcium channel blocking agents: | |
| Nifedipine | 30-180 |
| Nifedipine sustained release | 30-90 |
| Diltiazem | 90-360 |
| Diltiazem sustained release | 120-360 |
| Verapamil | 240-480 |
| Verapamil sustained release | 120-480 |
| Nicardipine | 30-120 |
| Nicardipine sustained release | 60-120 |
| Isradipine | 2.5-20 |
| Amlodipine | 5-10 |
| Felodipine sustained release | 5-20 |
| Direct vasodilators: | |
| Hydralazine | 20-300 |
| Minoxidil | 5-100 |
| Renin inhibitors | |
| Angiotensin II antagonists | |

A contemplated second antihypertensive agent formulation is prepared as is well known in the art for such agents. Most of the above agents are commercially available in immediate-release or extended-release formulations. Where a formulation type such as an extended-release formulation is not commercially available for a particular antihypertensive agent, the preparation of a desired formulation is well within the skill of the formulator's art.

This aspect of the invention is also suitable for once-per-day administration. It is therefore preferred that a single composition contain formulations of both active agents. Thus, in one exemplary composition, a delayed-release formulation of an aldosterone antagonist is present along with an immediate-release formulation of a second antihypertensive. Another exemplary composition contains a delayed-release formulation of an aldosterone antagonist and an extended-release formulation of a second antihypertensive.

Preparation of Aldosterone Antagonists

Eplerenone for use in a composition of the present invention can be prepared using the methods set forth in above-cited U.S. Pat. No. 4,559,332 and International Patent Publication No. WO 98/25948, particularly scheme 1 set forth in WO 98/25948. Spironolactone for use in a composition of the present invention can be prepared as described in U.S. Pat. No. 3,013,012.

Micronized and Nanoparticulate Aldosterone Antagonists

Although compositions of the invention are effective when prepared with eplerenone or spironolactone of a broad range of particle sizes, micronized or nanoparticulate aldosterone antagonists are preferred.

A D₉₀ particle size (defined elsewhere herein) of about 25 to about 400 μm can improve bioavailability of an aldosterone antagonist drug by comparison with larger particle sizes. Drug particles having a D₉₀ particle size of about 25 to about 400 μm are referred to herein as micronized particles.

Accordingly, the D₉₀ particle size of micronized eplerenone or spironolactone used as a starting material in preparing a contemplated composition is less than about 400 μm, preferably less than about 200 μm, more preferably less than about 150 μm, still more preferably less than about 100 μm, and still more preferably less than 90 μm. A particularly preferred D₉₀ micronized particle size is about 30 to about 110 μm, and more particularly still about 30 to about 50 μm. In other preferred embodiments, a particularly preferred D₉₀ particle size is about 50 to about 150 μm, still more preferably about 75 to about 125 μm. Micronized active agents so sized also typically exhibit a D₁₀ particle size (defined elsewhere herein) of less than 10 μm. For example, reducing the D₉₀ particle size of the drug from about 220 μm to about 90 μm in a contemplated composition can materially improve bioavailability of the drug.

Further reduction of particle size of an aldosterone antagonist drug to a D₉₀ particle size of about 10 μm to about 15 μm can also improve bioavailability of the drug.

Accordingly, in one embodiment, the D₉₀ particle size of an unformulated aldosterone antagonist used as a starting material in preparing a contemplated composition is less than about 15 μm, preferably less than about 10 μm, more preferably less than about 1 μm, still more preferably less than about 800 nm, more preferably still less than about 600 nm, and yet more preferably less than about 400 nm. In one embodiment, the D₉₀ particle size is about 10 nm to about 1 μm. In another embodiment, the D₉₀ particle size is about 100 nm to about 800 nm. In another embodiment, the D₉₀ particle size is about 200 nm to about 600 nm. In another embodiment, the D₉₀ particle size is about 400 nm to about 800 nm. Drug particles having a D₉₀ particle size less than about 15 μm are referred to herein as nanoparticles.

Dosage Form

Compositions of the present invention comprise an aldosterone antagonist such as a micronized or nanoparticulate form of eplerenone or spironolactone in association with one or more non-toxic, pharmaceutically-acceptable carriers, excipients and/or adjuvants (collectively referred to herein as “carrier materials”). Eplerenone is the preferred aldosterone antagonist and will be usually used hereinafter as exemplary of aldosterone antagonists in general.

The carrier materials used are pharmaceutically acceptable in the sense of being compatible with the other ingredients of the composition and not deleterious to the recipient. A composition of the invention is adapted for oral administration as a pill (tablet), a hard or soft capsule, coated granules, or any other form reasonably adapted for oral administration of a swallowable delayed-release composition.

Such a pharmaceutical composition is preferably made in the form of discrete dosage units, such as tablets or capsules, containing a predetermined amount of a delayed-release formulation of an aldosterone antagonist.

Delayed-release properties are provided by means of an enteric coating that survives passage of the dosage unit through the stomach into the intestinal tract where the coating provides delayed release of the aldosterone antagonist. In the case of tablets, the enteric coating typically and preferably surrounds each entire tablet. In the case of capsules containing beads or pellets, the enteric coating can surround individual beads or pellets. The portion of a tablet, bead or pellet surrounded by the enteric coating is herein referred to as a “core”. Preferably, the core comprises an immediate-release formulation of an aldosterone antagonist drug such as eplerenone. An extended-release core can be useful in some circumstances but is generally not preferred as it may not release sufficient of the drug prior to or during the relatively short duration of aldosterone acrophase. Furthermore, the combination of delayed release provided by the enteric coating and extended release provided by the core formulation may result in a clearance time for the drug that is too long to be compatible with once-a-day administration.

Carrier Materials

Carrier materials or excipients useful in core formulations of compositions of the invention preferably are water-soluble or water-dispersible and include materials having wetting properties to offset the low aqueous solubility and hydrophobicity of the aldosterone antagonist, e.g., eplerenone. Such carrier materials include diluents, disintegrants, binding agents and adhesives, wetting agents, lubricants, anti-adherent agents and/or other carrier materials including buffering agents.

In addition, delayed-release formulations of the invention have, surrounding the core, an enteric coating that comprises additional excipients or carrier materials as described below.

Diluents

A composition of the invention optionally comprises one or more pharmaceutically acceptable diluents as a carrier material. Suitable diluents can include, either individually or in combination, lactose, including lactose USP, anhydrous lactose USP and spray-dried lactose USP; starch, including starch USP and directly compressible starch; mannitol; sorbitol; dextrose monohydrate; microcrystalline cellulose; dibasic calcium phosphate dihydrate; sucrose-based diluents, including confectioner's sugar and sugar spheres NF; monobasic calcium sulfate monohydrate; calcium sulfate dihydrate; calcium lactate trihydrate (e.g., calcium lactate trihydrate granular NF); dextrates (e.g., Emdex™); Celutab™; dextrose (e.g., Cerelose™); inositol; hydrolyzed cereal solids such as Maltron™ products and Mor-Rex™; amylose; Rexcel™; powdered cellulose (e.g., Elcema™); calcium carbonate; glycine; bentonite; polyvinylpyrrolidone; and the like.

Such diluents, if present, constitute in total about 5% to about 99%, preferably about 25% to about 90%, and more preferably about 40% to about 80%, by weight of the core. The diluent or diluents selected for tablets preferably exhibit suitable compressibility and pre-compression flow properties.

Microcrystalline cellulose (e.g., Avicel® PH 101) and lactose, either individually or in combination, are preferred diluents for use in tablets. Both diluents are chemically compatible with eplerenone or spironolactone. The use of extragranular microcrystalline cellulose (that is, microcrystalline cellulose added to a wet granulated composition after a drying step) in addition to intragranular microcrystalline cellulose (that is, microcrystalline cellulose added to the composition during or before a wet granulation step) can be used to improve tablet hardness and/or disintegration time. Lactose, especially lactose monohydrate, is particularly preferred. Lactose typically provides core formulations having suitable eplerenone release rates, stability, pre-compression flowability, and drying properties at a relatively low diluent cost.

Sugar spheres, e.g., sugar spheres NF, are a preferred diluent for use in encapsulated beads or pellets. Here, a desired amount of the aldosterone antagonist is coated upon the sugar spheres, with an outer coating of a polymeric material as described below that provides the delayed-release.

Disintegrants

Compositions of the invention optionally comprise one or more pharmaceutically acceptable disintegrants as a carrier material, particularly for tablet formulations. Suitable disintegrants include, either individually or in combination, starches; sodium starch glycolate; clays (such as Veegum™ HV); celluloses (such as purified cellulose, methylcellulose, sodium carboxymethylcellulose and carboxymethylcellulose); alginates; pregelatinized corn starches (such as National 1551 and National 1550); croscarmellose sodium; crospovidone; and gums (such as agar, guar, locust bean, karaya, pectin, and tragacanth gums). Disintegrants can be added at any suitable step during the preparation of the core formulation, particularly prior to granulation or during a lubrication step prior to compression.

Such disintegrants, if present, constitute in total about 0.5% to about 30%, preferably about 1% to about 10%, and more preferably about 2% to about 6%, by weight of the core. Croscarmellose sodium is a preferred disintegrant for tablet formulations, suitably in an amount of about 1% to about 10%, preferably about 2% to about 6%, and more preferably about 5%, by weight of the core.

Binding Agents and Adhesives

Compositions of the invention optionally comprise one or more pharmaceutically acceptable binding agents or adhesives as a carrier material. Such binding agents and adhesives preferably impart sufficient cohesion to powders to permit normal processing operations such as sizing, lubrication, compression and packaging, but still permit the core to disintegrate and the drug to become available for absorption upon erosion or disintegration of the delayed-release coating.

Suitable binding agents and adhesives include, either individually or in combination, such binding agents and adhesives as acacia; tragacanth; sucrose; gelatin; glucose; starch; cellulose materials such as, but not limited to, methylcellulose and sodium carboxymethylcellulose (e.g., Tylose™); alginic acid and salts of alginic acid; magnesium aluminum silicate; polyethylene glycol; guar gum; polysaccharide acids; bentonites; polyvinylpyrrolidone (povidone); alkali and alkaline earth metal polymethacrylates; hydroxypropyl methylcellulose (HPMC); hydroxypropylcellulose (e.g., Klucel™); ethylcellulose (e.g., Ethocel™); and pregelatinized starch (such as National 1511 and Starch 1500). Such binding agents and/or adhesives, if present, constitute in total about 0.5% to about 25%, preferably about 0.75% to about 15%, and more preferably about 1% to about 10%, by weight of the core.

HPMC is a preferred binding agent used to impart cohesive properties to a powder of eplerenone in a tablet formulation. HPMC is suitably present in an amount of about 0.5% to about 10%, preferably about 1% to about 8%, and more preferably about 2% to about 4%, by weight of the core. Low molecular weight HPMC having a viscosity of about 2 cPs to about 8 cPs typically can be used, although viscosities of about 2 cPs to about 6 cPs are preferred, particularly viscosities of about 2 cPs to about 4 cPs. Viscosities are measured as a 2 percent solution in water at 20° C.

HPMCs vary in the degree of substitution of available hydroxyl groups on the cellulosic backbone by methoxyl groups and by hydroxypropoxyl groups. With increasing hydroxypropoxyl substitution, the resulting HPMC becomes more hydrophilic in nature. It is preferred in core formulations of the present invention to use HPMCs having about 15% to about 35% methoxyl substitution, and up to about 15%, more preferably about 2% to about 12%, hydroxypropyl substitution.

For a composition that comprises a delayed-release formulation of an aldosterone antagonist in the form of coated beads such as those based on sugar spheres, povidone is a preferred binding agent or adhesive and is typically present at about 0.5% to about 2% of the core formulation. In this instance, the povidone can be thought of as an adhesive that entraps and glues the aldosterone antagonist to the sugar spheres.

Wetting Agents

Eplerenone and spironolactone, even in micronized or nanoparticulate form, are largely insoluble in aqueous solution. Accordingly, compositions of the present invention optionally comprise one or more wetting agents as a carrier material, particularly for tablet formulations. Such wetting agents are preferably selected to maintain the eplerenone or spironolactone in close association with water, a condition that is believed to improve the bioavailability of the drug.

Suitable wetting agents include, either individually or in combination, oleic acid; glyceryl monostearate; sorbitan monooleate; sorbitan monolaurate; triethanolamine oleate; polyoxyethylene sorbitan monooleate; polyoxyethylene sorbitan monolaurate; sodium oleate; and sodium lauryl sulfate. Wetting agents that are anionic surfactants are preferred. Such wetting agents, if present, constitute in total about 0.1% to about 15%, preferably about 0.25% to about 10%, and more preferably about 0.5% to about 5%, by weight of the core.

Sodium lauryl sulfate is a preferred wetting agent for tablet formulations. Sodium lauryl sulfate is suitably present in an amount of about 0.25% to about 7%, preferably about 0.4% to about 4%, and more preferably about 0.5% to about 2%, by weight of the composition.

Lubricants

Compositions of the invention optionally comprise one or more lubricants and/or glidants as a carrier material. Suitable lubricants and/or glidants include, either individually or in combination, glyceryl tribehenate (e.g., Compritol™ 888); stearates (e.g., magnesium, calcium and sodium stearates); stearic acid; hydrogenated vegetable oils (e.g., Sterotex™); talc; waxes; Stearowet™; boric acid; sodium benzoate and sodium acetate; sodium chloride; DL-leucine; polyethylene glycols (e.g., Carbowax™ 4000 and Carbowax™ 6000); sodium oleate; sodium benzoate; sodium acetate; sodium lauryl sulfate; sodium stearyl fumarate (e.g., Pruv™); and magnesium lauryl sulfate.

Such lubricants, if present, constitute in total about 0.1% to about 10%, preferably about 0.2% to about 8%, and more preferably about 0.25% to about 5%, by weight of the core. Magnesium stearate is a preferred lubricant used to reduce friction between the equipment and granulation during compression of tablet formulations.

Anti-Adherent Agents or Glidants

Compositions of the invention optionally comprise one or more anti-adherent agents or glidants as a carrier material. Suitable anti-adherents or glidants include, either individually or in combination, talc, corn starch, colloidal silica (e.g., Cab-O-Sil™), Syloid™, DL-leucine, sodium lauryl sulfate, and stearates. Such anti-adherents or glidants, if present, constitute in total about 0.1% to about 15%, preferably about 0.25% to about 10%, and more preferably about 0.5% to about 5%, by weight of the core.

Talc is a preferred anti-adherent or glidant agent used to reduce formulation sticking to equipment surfaces and also to reduce static in the blend. Talc is suitably present at about 0.1% to about 10%, preferably about 0.25% to about 5%, and more preferably about 0.5% to about 2%, by weight of the composition.

Other carrier materials (such as colorants, flavors and sweeteners) and modes of administration are known in the pharmaceutical art and can be used in preparation of compositions of the present invention.

Preferred Core Formulations

In one embodiment of the present invention, an immediate-release core comprises eplerenone or spironolactone in a desired amount and one or more cellulosic carrier materials. The term “cellulosic carrier materials” embraces carrier materials comprising cellulose or a cellulose derivative such as purified cellulose; microcrystalline cellulose; and alkyl celluloses and their derivatives and salts (e.g., methylcellulose, sodium carboxymethylcellulose, carboxymethylcellulose, croscarmellose sodium, hydroxypropylcellulose, hydroxypropyl methylcellulose and the like). Preferably, at least one carrier material is a cellulosic material selected from the group consisting of (C₁-C₆ alkyl)celluloses and their derivatives and salts. Still more preferably, this cellulosic material is selected from the group consisting of hydroxyalkyl alkylcelluloses and their derivatives and salts. Still more preferably, this cellulosic material is selected from the group consisting of hydroxy(C₂-C₄ alkyl) (C₁-C₄ alkyl)celluloses and their derivatives and salts.

The core formulation preferably further comprises one or more carrier materials selected from the group consisting of diluents, disintegrants, binding agents, wetting agents, lubricants and anti-adherent agents. More preferably, the core comprises one or more carrier materials selected from the group consisting of lactose, microcrystalline cellulose, croscarmellose sodium, hydroxypropyl methylcellulose, sodium lauryl sulfate, magnesium stearate, and talc. Still more preferably, the core comprises lactose monohydrate, microcrystalline cellulose, croscarmellose sodium and hydroxypropyl methylcellulose. Still more preferably, such a core further comprises one or more of the carrier materials sodium lauryl sulfate, magnesium stearate, and talc.

The individual pharmaceutically acceptable carrier materials described in the above embodiment optionally can be replaced with other suitable carrier materials if desired. Acceptable substitute carrier materials are chemically compatible both with the aldosterone antagonist and with the other carrier materials. Although other diluents, disintegrants, binding agents and adhesives, wetting agents, lubricants and/or anti-adherent or glidant agents can be employed, pharmaceutical compositions comprising micronized eplerenone, lactose, microcrystalline cellulose, croscarmellose sodium and hydroxypropyl methylcellulose, and, optionally, sodium lauryl sulfate, magnesium stearate and/or talc, provide a desirable combination of pharmacokinetic, chemical and physical properties.

C₁₂-C₂₄ Fatty Acid

A C₁₂-C₂₄ fatty acid can also be present in a core formulation to assist in enhancing bioavailability of the aldosterone antagonist. A similar effect was reported in U.S. Pat. No. 5,391,377 for different active agents.

A contemplated fatty acid can be saturated or unsaturated. An exemplary saturated fatty acid is stearic acid. An exemplary monounsaturated fatty acid is oleic acid, whereas illustrative polyunsaturated fatty acids are linoleic and linolenic acids. The fatty acid can be present at a weight ratio relative to the aldosterone antagonist at about 10:1 to about 1:10. Preferably, the weight ratio is about 5:1 to about 1:1.

Medicament Composition

The aldosterone antagonist is combined with one or usually more of the above ingredients to form a core formulation. The core formulation can also include a second antihypertensive agent, although it is preferred that where a second antihypertensive agent is present it be separately compounded into a formulation other than the delayed-release formulation comprising the aldosterone antagonist, and the two resulting formulations then be combined to form a medicament composition.

As used herein, the term “core” refers to the formulation, containing an aldosterone antagonist such as eplerenone and various carriers, that is enclosed in a delayed-release coating as described below. The core typically can be about 200 μm to 1700 μm in diameter in the case of beads or pellets, but can be larger in the case of tablets.

Prior to being enterically coated, a contemplated formulation is present in the form of uncoated or naked cores. In one embodiment, the cores are relatively large and are in the form of pills or tablets that have a longest dimension of about 2 mm to about 5 mm. In another embodiment, the cores are even larger, in the form of lozenges with a longest dimension of up to about 10 mm. In a still further, more preferred embodiment, the cores are in the form of generally spherical beads having a diameter of about 1 mm or preferably less, for example about 0.2 mm to about 0.8 mm. Such beads can be used after coating alone or within a capsule, which is preferred.

In one embodiment, substantially all beads in a capsule are coated to provide delayed-release properties. In another embodiment, some beads are enterically coated as described below and others, within the same capsule, are uncoated or coated with a non-delayed-release coating. In this embodiment the beads having no delayed-release coating can contain a second antihypertensive agent. In yet another embodiment, a single capsule contains beads having a variety of delayed-release coatings providing a range of delayed-release periods.

Enteric Coatings

A delayed-release formulation of an aldosterone antagonist according to the invention has an outer enteric coating providing a hydratable diffusion barrier that is a primary agent responsible for providing the delayed-release properties. The term “enteric coating” herein embraces any coating material having the required properties set out herein.

The hydratable enteric coating diffusion barrier preferably comprises one or more film-forming polymers that are acid- and water-insoluble under stomach and intestinal conditions and preferably includes additives that help control the rate of hydration and permeability of the diffusion barrier.

The composition of the polymeric material as well as the amount of material that is utilized affects whether a particular formulation provides the desired dissolution or release characteristics.

In one embodiment, the polymeric coating is produced from polymerized acrylates or copolymers of acrylic acid and methacrylic acid or esters of either monomer (hereinafter “polymerized acrylates”).

Polymerized acrylates are known in the art and are available from many commercial sources. Examples of such polymerized acrylates include poly(methyl methacrylate), poly(ethyl methacrylate), poly(butyl methacrylate), poly(isobutyl methacrylate), poly(phenyl methacrylate) and the like. The amount of polymerized acrylates contained within the polymeric coating can vary. Typically, the polymeric coating contains about 10% to about 50%, preferably about 15% to about 35%, by weight of polymerized acrylate.

The polymeric coating of the delayed-release formulation can also be prepared from one of the organosiloxane oral coating materials known in the art such as polydimethylsiloxane, polydiethylsiloxane, and the like. The organosiloxane oral coating material can be used in similar quantities to the polymerized acrylate set out above. A greater amount of polymeric coating is used when a longer delay prior to active agent release is desired, whereas a smaller amount of polymeric coating is used when a shorter delay before release is desired.

Preferred water-insoluble film-forming polymers include an aqueous dispersion of fully esterified acrylic resins sold as Eudragit™ NE30D available from Rohm Pharma GmbH of Weiterstadt, Germany, aqueous dispersions of ethylcellulose such as Aquacoat™ ethylcellulose emulsion, available from FMC Corp. of Chicago, Ill. or Surelease™, available from Colorcon of West Point, Pa. Polymers dissolved in organic solvents can also be used.

Other preferred polymerized acrylates are those that are water-insoluble, slightly water-permeable copolymers of an acrylic acid lower alkyl ester and a methacrylic acid lower alkyl ester in which some ester moieties are further substituted with a tri(alkyl)ammonium group. The tri(alkyl)ammonium group is typically present in the range of about 1:30 to about 1:50 relative to the amount of neutral ester present. The alkyl portion of a tri(alkyl)ammonium group is a C₁-C₄ alkyl group, with C₁ alkyl (methyl) being preferred. One such preferred copolymer is a copolymer of ethyl acrylate and methyl methacrylate that contains trimethylammonium ethyl methacrylate in a range of about 1:40 relative to the neutral monomers. This copolymer is commercially available from Rohm Pharma GmbH under the trade name Eudragit™ RS.

A polymeric enteric coating can additionally or alternatively contain a water-insoluble, freely water-permeable copolymer of an acrylic acid lower alkyl ester and a methacrylic acid lower alkyl ester in which some ester moieties are further substituted with a tri(alkyl)ammonium group, as noted above. in this case, the tri(alkyl)ammonium group is present in an amount of about 1:20 relative to the amount of methacrylic and acrylic monomer utilized. One such preferred copolymer is a copolymer of ethyl acrylate and methyl methacrylate that contains trimethylammonium ethyl methacrylate in a ratio of about 1:20 to the neutral monomers. This copolymer is commercially available from Rohm Pharma GmbH under the trade name Eudragit™ RL. The ratio of water-insoluble, slightly water-permeable acrylate to water-insoluble, freely water-permeable acrylate is 100:zero to about 70:30 and more preferably about 95:5.

Additional substances that can be used that are less permeable to water include ethylcellulose (noted above), cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride) or polyurethane or a mixture of any two or more of these. Suitable naturally occurring polymers or resins that are less permeable to water include shellac, chitosan, gum juniper or a mixture of two or more of these.

The coating layer can comprise a mixture of polymers, synthetic and/or naturally occurring, that are freely permeable, slightly permeable, water-soluble or water-insoluble, and polymers whose permeability and/or solubility is not affected by pH value. In addition to those referred to above, such suitable polymers for inclusion into the coating layer include Eudragit™ S, Eudragit™ L, Eudragit™ E, polyvinyl alcohol and polyvinylpyrrolidone.

Additives that control the rate of hydration and permeability of the enteric coating diffusion barrier include fully esterified acrylic resins containing a quaternary amine side chain, anionic surfactants, lubricants, plasticizers, inert water-soluble materials, and mixtures thereof. In a preferred embodiment, these additives comprise fully esterified acrylic resins containing quaternary amine side chains such as Eudragit™ RS30D and RL30D available from Rohm Pharma GmbH, sodium lauryl sulfate, magnesium stearate, citric acid, simethicone, and mixtures thereof. The use of acrylic resins such as Eudragit™ RS30D and RL30D increases the permeability of the diffusion barrier and permits thicker enteric coating diffusion barriers to be used to provide longer time delays.

In addition to the polymers described above, the coating layer typically includes a lubricant and a wetting agent or surfactant. Preferably, the lubricant is talc and the wetting agent is sodium lauryl sulfate. Suitable alternatives for sodium lauryl sulfate can include agents such as acacia, benzalkonium chloride, cetomacrogol emulsifying wax, cetostearyl alcohol, cetyl alcohol, cholesterol, diethanolamine, docusate sodium, sodium stearate, emulsifying wax, glyceryl monostearate, hydroxypropyl cellulose, lanolin alcohols, lecithin, mineral oil, monoethanolamine, poloxamer, polyoxyethylene alkyl ethers, polyoxyethylene castor oil derivatives, polyoxyethylene sorbitan fatty acid esters, polyoxyethylene stearates, propylene glycol alginate, sorbitan esters, stearyl alcohol and triethanolamine, or a mixture of any two or more of the foregoing. Surfactants are preferably used in an amount of zero to 2% of the film-forming polymer weight, and function to increase both the permeability and hydration rate of the coating.

Suitable alternatives for talc that can be included in the coating are calcium stearate, colloidal silicon dioxide, glycerin, magnesium stearate, mineral oil, polyethylene glycol, and zinc stearate, aluminum stearate or a mixture of any two or more of the foregoing. Magnesium stearate decreases the hydration rate, increases the hydrated permeability of the diffusion barrier, and also prevents the cores from agglomerating during processing.

A plasticizing agent is preferably included in the enteric coating to improve elasticity and stability of the polymer film and to prevent changes in polymer permeability over prolonged storage. Such changes could affect the drug release properties. Suitable conventional plasticizing agents include acetylated monoglycerides, acetyl tributyl citrate, acetyl triethyl citrate, castor oil, citric acid esters, dibutyl phthalate, dibutyl sebacate, diethyl oxalate, diethyl malate, diethyl fumarate, diethyl phthalate, diethyl succinate, diethyl malonate, diethyl tartrate, dimethyl phthalate, glycerin, glycerol, glyceryl triacetate, glyceryl tributyrate, mineral oil and lanolin alcohols, petrolatum and lanolin alcohols, phthalic acid esters, polyethylene glycols, propylene glycol, rape oil, sesame oil, triacetin, tributyl citrate, triethyl citrate, or a mixture of any two or more of the foregoing. Preferred plasticizers are tributyl citrate, triethyl citrate and acetyl tributyl citrate. The amount of plasticizer utilized can vary, but is typically zero to about 40%, preferably about 5% to about 15%, by weight of the polymeric coating.

Commercially available polymeric solutions and/or suspensions can also be used. These solutions and/or suspensions can optionally contain endogenous plasticizing agents to improve the polymer characteristics of the coating. Examples of such solutions and/or suspensions include Eudragit™ RL 30D, Eudragit™ L 30D, Eudragit™ E 12.5, Eudragit™ RL 12.5 P, Eudragit™ RS 12.5, Aquacoat™, and Surelease™. Aquacoat™ is an aqueous polymeric dispersion of ethylcellulose and contains sodium lauryl sulfate and cetyl alcohol. Surelease™ is an aqueous polymeric dispersion of ethylcellulose and contains dibutyl sebacate, oleic acid, ammoniated water and fumed silica.

In addition to preferably containing polymerized acrylate and optionally a plasticizer, the polymeric coating can contain conventional excipients including antifoaming agents such as simethicone, in the range of zero to about 2% by weight of the polymeric coating. The coating can also contain an anti-adherent such as talc at zero to about 70%, preferably about 25% to about 35% by weight of the polymeric coating.

A sufficient quantity of the polymeric coating is utilized to substantially envelop the central core in order to give the formulation so formed the desired delayed-release characteristics. The exact quantity of polymeric coating can vary depending upon the composition of the central core and the manner in which it is produced. For example, factors that can affect the amount of polymeric coating required include the size of the central core, the size of any inert carrier used in producing the central core, whether the central core was produced via compression or granulation or build-up, the composition of the binding agent, and the amount of binding agent utilized. The exact amount required can be calculated by one skilled in the art utilizing the dissolution profile taught herein for the delayed-release particle. Typically, based upon the total weight of the delayed-release formulation after proper drying, the polymeric coating is present in the quantity of about 15% to about 50%, more preferably about 20% to about 30%, for example about 25%, by weight, the remainder of the weight being contributed by the central core.

Method to make Celecoxib Coated Beads

The polymeric enteric coating can be applied to the central cores using methods and techniques known in the art. Typically a suspension, emulsion, or solution of the polymeric coating is prepared as is known in the art. The amount of the resulting fluidized polymeric coating required in the coating process can be readily calculated depending upon the amount of polymeric coating desired in the dried formulation.

The fluidized polymeric coating can be applied to the central core by a number of coating techniques known in the art. Examples of suitable coating devices include fluid bed coaters, pan coaters, and the like. After the polymeric coating has been applied to the central core, the particles are then dried. The coating and drying processes are continued as required until the particles have the desired dissolution profile described herein.

The process described below is an illustrative method to make eplerenone enteric coated beads.

1. Mixing and granulating: Eplerenone and diluents, preferably lactose and/or microcrystalline cellulose, are mixed and granulated by the following illustrative process. Eplerenone is added to a mixture of lactose and microcrystalline cellulose (e.g., Avicel™ PH-101 and/or Avicel™ RC-581 or Avicel™ RC-591) in a total amount of 1000-4000 g and are dry-mixed in a high shear mixer (e.g., Niro-Fielder mixer) at a high mixing speed for 2-5 minutes. Water (300-700 grams) is added and the mass is granulated for 2-5 minutes at high speed.

2. Extrusion: Extrusion of the resulting material can be performed for example in a NICA E-140 extruder (Lejus Medical AB, Sweden) through a perforated screen with drilled orifices of 0.25-1.0 mm diameter. The speed of the agitator and the feeder are preferably set on the lowest values.

3. Spheronization: Spheronization of the resulting extrudate can be conducted in a NICA marumerizer (Ferro Mecano AB, Sweden). The speed of the marumerizer plate is preferably adjusted to 500-10,000 rpm. The spheronization continues for 2-10 minutes, with about 1000 g wet extrudate on the plate at each run.

4. Drying: Drying of the resulting spheronized beads can be performed in a fluidized bed dryer (e.g., Aeromatic AG West Germany) at an inlet temperature of 50-90° C. A net device can be placed in the top of the fluidized bed to avoid loss of beads to the cyclone output. The batch is preferably divided into sub-batches of 200-800 g. Each sub-batch is dried for 10-60 minutes at an air volume of 100-400 m³/h in order to obtain individual beads rather than aggregates. If necessary, the sub-batches are then mixed and the whole batch dried for 5-30 minutes to an end product temperature of 40-60° C. A yield of dry beads of 1600-2000 g can be expected.

5. Sizing: Sizing of the resulting dry beads can be performed using analytical sieves. Two sieves are selected from a set of sieve sizes, for example of 850 μm, 600 μm, 425 μm, 300 μm, 250 μm and 180 μm. A preferred pair of sieves for sizing beads of the present invention is 425 μm and 180 μm.

6. Coating: Eplerenone beads manufactured as above are coated with an enteric coating to prepare delayed-release formulations of the present invention. For example, Surelease™ or Eudragit™ RS can be applied as a 10-20% by weight solids dispersion, using spray coating equipment (e.g., Wurster). The spray gun is mounted at a height of 0.25 cm to 5 cm over the bottom of the bed. Ethanol/MIBK mixture is pumped through the system prior to the start of the coating operation. Eplerenone beads prepared as above are loaded. The beads are pre-heated at 50-80° C. with an air velocity of 100-400 m³/h for 30-90 minutes. The coating is applied using the following process parameters: atomizing pressure 1.0-3.0 bar, air temperature 50-80° C., air velocity 100-400 m³/h and solution flow about 10-80 ml/minute.

7. Encapsulating: The coated beads manufactured as above, optionally together with uncoated beads, are encapsulated by a conventional encapsulation process.

Dissolution Profile

The compositions of the present invention preferably are formulated to provide an in vitro dissolution profile in a type 2 dissolution apparatus (paddle) according to U.S. Pharmacopeia 24 (2000), Test No. 711, at 50 rpm, with an aqueous dissolution medium containing 1% SDS at 37° C., in which:
(a) zero to about 20%, preferably zero to about 10%, of the aldosterone antagonist is dissolved at about 4 hours; and
(b) about 50% to 100%, preferably about 70% to 100%, of the aldosterone antagonist is dissolved within a period of about 3 hours that begins at a time t about 4 to about 12 hours, preferably about 5 to about 10 hours, after initiation of dissolution.

Particularly preferred are compositions wherein release of the aldosterone antagonist is delayed for at least about 6 hours. Accordingly, in a preferred embodiment, compositions are formulated to provide an in vitro dissolution profile in the designated test in which:
(a) zero to about 20%, preferably zero to about 10%, of the aldosterone antagonist is dissolved at about 6 hours; and
(b) about 50% to 100%, preferably about 70% to 100%, of the aldosterone antagonist is dissolved within a period of about 3 hours that begins at a time t about 6 to about 12 hours, preferably about 6 to about 10 hours, after initiation of dissolution.

It is further preferred that zero to about 5% of the aldosterone antagonist is dissolved at about 3 hours after initiation of dissolution.

Granulation Particle Size and Flow Properties

Although compositions of the invention can be prepared, for example, by direct encapsulation or direct compression, they preferably are wet granulated prior to encapsulation or compression. Wet granulation, among other advantages, densifies the compositions resulting in improved flow properties, improved compression characteristics and easier metering or weight dispensing of the final compositions. The average particle size of the granulation preferably permits for convenient handling and processing and, for tablets, permits formation of a directly compressible mixture that forms pharmaceutically acceptable tablets. The desired tap and bulk densities of the granulation are normally about 0.3 g/ml to about 1.0 g/ml, preferably about 0.4 g/ml to about 0.8 g/ml.

Hardness

For tablet formulations, a composition in an amount sufficient to make a uniform batch of tablets is subjected to tableting in a conventional production-scale tableting machine at normal compression pressure (for example, about 1 kN to about 50 kN). Any tablet hardness convenient with respect to handling, manufacture, storage and ingestion may be employed. Hardness in the range of about 3.5 kP to about 22 kP is typically acceptable, with about 3.5 kP to about 9 kP preferred for 25 mg tablets, about 5 kP to about 13 kP preferred for 50 mg tablets, and about 8 kP to about 22 kP preferred for 100 mg tablets. The mixture, however, is not to be compressed to such a degree that there is subsequent difficulty in achieving hydration when exposed to intestinal fluid following erosion or disintegration of the enteric coating.

Friability

For tablet formulations, tablet friability preferably is less than about 0.8%, more preferably less than 0.4%, in a standard test.

Illustrative Core Formulations

Exemplary preferred compositions of core formulations (i.e., prior to being enterically coated) of an aldosterone antagonist are provided hereinafter, illustratively using eplerenone as the aldosterone antagonist.

The term “% by weight” as used herein means the weight percent of a specified ingredient based upon the total weight of all ingredients of the core formulation.

A formulation of one embodiment comprises micronized or nanoparticulate eplerenone in an amount sufficient to provide a desired daily dosage of eplerenone, that is, about 10 mg to about 400 mg, preferably about 10 mg to 200 mg, more preferably about 20 mg to 100 mg, still more preferably about 20 mg to 75 mg, and still more preferably about 25 mg to about 50 mg. A once-a-day tablet or capsule thus preferably contains eplerenone in an amount, for example, of about 25 mg to about 50 mg. Preferably also, one batch of a formulation prior to tableting or encapsulation can be used to prepare tablets or capsules of different strengths by compressing the formulation in different tablet sizes, or by encapsulating the formulation in different capsule sizes or using different capsule fill weights. Although the amount of eplerenone preferably is within the ranges previously discussed, formulations of the invention also can be useful for administration of an amount of eplerenone falling outside the disclosed dosage ranges.

In one embodiment, a contemplated medicament composition comprises:
about 1% to about 90% by weight of micronized or nanoparticulate eplerenone;
about 5% to about 90% by weight of lactose;
about 5% to about 90% by weight of microcrystalline cellulose; and
about 0.5% to about 10% by weight of hydroxypropyl methylcellulose.

The medicament composition just described optionally can additionally comprise:
about 1% to about 10% by weight of croscarmellose sodium;
about 0.1% to about 7% by weight of sodium lauryl sulfate;
about 0.1% to about 10% by weight of talc; and/or
about 0.1% to about 10% by weight of magnesium stearate.

More preferably, the medicament composition comprises:
about 19% to about 40% by weight of micronized or nanoparticulate eplerenone;
about 32% to about 52% by weight of lactose;
about 8% to about 28% by weight of microcrystalline cellulose;
about 1% to about 10% by weight of croscarmellose sodium; and
about 1% to about 8% by weight of hydroxypropyl methylcellulose.

The medicament composition just described optionally can additionally comprise:
about 0.1% to about 7% by weight of sodium lauryl sulfate;
about 0.1% to about 10% by weight of talc; and
about 0.1% to about 10% by weight of magnesium stearate.

Preferably, the hydroxypropyl methylcellulose has a viscosity of about 2 cPs to about 8 cPs, more preferably about 2 cPs to about 6 cPs.

Still more preferably, the medicament composition comprises:
about 24% to about 35% by weight of micronized or nanoparticulate eplerenone;
about 37% to about 47% by weight of lactose;
about 13% to about 23% by weight of microcrystalline cellulose;
about 2% to about 6% by weight of croscarmellose sodium; and
about 2% to about 4% by weight of hydroxypropyl methylcellulose.

The medicament composition just described optionally can additionally comprise:
about 0.25% to about 4% by weight of sodium lauryl sulfate;
about 0.1% to about 5% by weight of talc; and
about 0.25% to about 5% by weight of magnesium stearate.

Preferably, the hydroxypropyl methylcellulose has a viscosity of about 2 cPs to about 6 cPs.

Still more preferably, the medicament composition comprises:
about 28% to about 31% by weight of micronized or nanoparticulate eplerenone;
about 41% to about 43% by weight of lactose monohydrate;
about 17% to about 19% by weight of microcrystalline cellulose;
about 4.5% to about 5.5% by weight of croscarmellose sodium; and
about 2.5% to about 3.5% by weight of hydroxypropyl methylcellulose.

The medicament composition just described optionally can additionally comprise:
about 0.5% to about 1.5% by weight of sodium lauryl sulfate;
about 0.5% to about 1.5% by weight of talc; and
about 0.25% to about 0.75% by weight of magnesium stearate.

Preferably, the hydroxypropyl methylcellulose has a viscosity of about 2 cPs to about 4 cPs.

In another embodiment, a contemplated medicament composition comprises:
about 10 mg to about 200 mg of micronized or nanoparticulate eplerenone;
about 30 mg to about 150 mg of lactose;
about 10 mg to about 70 mg of microcrystalline cellulose; and
about 1 mg to about 15 mg of hydroxypropyl methylcellulose.

The medicament composition just described optionally can additionally comprise:
about 1 mg to about 25 mg of croscarmellose sodium;
about 0.25 mg to about 5 mg of sodium lauryl sulfate;
about 0.5 mg to about 5 mg of talc; and
about 0.5 mg to about 3 mg of magnesium stearate.

Preferably, the hydroxypropyl methylcellulose has a viscosity of about 2 cPs to about 8 cPs, more preferably about 2 cPs to about 6 cPs.

In another embodiment, a medicament composition comprises:
about 20 to about 50 mg of micronized or nanoparticulate eplerenone;
about 34 mg to about 38 mg of lactose;
about 14 mg to about 17 mg of microcrystalline cellulose;
about 3 mg to about 6 mg of croscarmellose sodium; and
about 1 mg to about 4 mg of hydroxypropyl methylcellulose.

The medicament composition just described optionally can additionally comprise:
about 0.25 mg to about 1.5 mg of sodium lauryl sulfate;
about 0.25 mg to about 1.5 mg of talc; and
about 0.1 mg to about 1 mg of magnesium stearate.

Preferably, the hydroxypropyl methylcellulose has a viscosity of about 2 cPs to about 6 cPs. The composition is preferably in the form of a tablet.

In another embodiment, the medicament composition comprises:
about 25 mg to about 50 mg of micronized or nanoparticulate eplerenone;
about 70 mg to about 73 mg of lactose;
about 29 mg to about 33 mg of microcrystalline cellulose;
about 6 mg to about 10 mg of croscarmellose sodium; and
about 4 mg to about 6 mg of hydroxypropyl methylcellulose.

The medicament composition just described optionally can additionally comprise:
about 1 mg to about 2.5 mg of sodium lauryl sulfate;
about 1 mg to about 2.5 mg of talc; and
about 0.5 mg to about 1.5 mg of magnesium stearate.

Preferably, the hydroxypropyl methylcellulose has a viscosity of from about 2 cPs to about 6 cPs. The composition is preferably in the form of a tablet.

In another embodiment, the medicament composition comprises:
about 40 mg to about 100 mg of micronized or nanoparticulate eplerenone;
about 141 mg to about 145 mg of lactose;
about 60 mg to about 64 mg of microcrystalline cellulose;
about 16 mg to about 18 mg of croscarmellose sodium; and
about 9 mg to about 11 mg of hydroxypropyl methylcellulose.

The medicament composition just described optionally can additionally comprise:
about 3 mg to about 4 mg of sodium lauryl sulfate;
about 3 mg to about 4 mg of talc; and
about 1 mg to about 2 mg of magnesium stearate.

Preferably, the hydroxypropyl methylcellulose has a viscosity of from about 2 cPs to about 6 cPs. The composition is preferably in the form of a tablet.

In another embodiment, the medicament composition comprises lactose, microcrystalline cellulose, croscarmellose sodium, hydroxypropyl methylcellulose, sodium lauryl sulfate, talc and magnesium stearate.

In another embodiment, the medicament composition comprises:
about 20 mg to about 200 mg of micronized or nanoparticulate eplerenone;
about 48 mg to about 242 mg of lactose; and
about 2 mg to about 56 mg of microcrystalline cellulose.

The medicament composition just described optionally can additionally comprise:
about 0.25 mg to about 18 mg of croscarmellose sodium;
about 0.1 mg to about 5 mg of sodium lauryl sulfate;
about 0.5 mg to about 8 mg of talc;
about 0.1 mg to about 5 mg of magnesium stearate; and
about 0.1 mg to about 5 mg of colloidal silicon dioxide.

In another embodiment, the medicament composition comprises:
about 20 to about 50 mg of micronized or nanoparticulate eplerenone;
about 56 mg to about 60 mg of lactose;
about 9.5 mg to about 13.5 mg of microcrystalline cellulose; and
about 0.5 mg to about 3.5 mg of croscarmellose sodium.

The medicament composition just described optionally can additionally comprise:
about 0.1 mg to about 1.5 mg of sodium lauryl sulfate;
about 0.25 mg to about 4.5 mg of talc;
about 0.1 mg to about 1.5 mg of magnesium stearate; and
about 0.1 to about 2.5 weight percent colloidal silicon dioxide.

The composition of this embodiment is preferably in the form of a capsule.

In another embodiment, the medicament composition comprises:
about 25 mg to about 50 mg of micronized or nanoparticulate eplerenone;
about 114 mg to about 118 mg of lactose;
about 21 mg to about 25 mg of microcrystalline cellulose; and
about 2 mg to about 6 mg of croscarmellose sodium.

The medicament composition just described optionally can additionally comprise:
about 1 to about 2.5 mg of sodium lauryl sulfate;
about 2 to about 8 mg of talc;
about 0.25 mg to about 1.5 mg of magnesium stearate; and
about 0.1 to about 3 weight percent of colloidal silicon dioxide.

The composition of this embodiment is preferably in the form of a capsule.

In another embodiment, the medicament composition comprises:
about 98 mg to about 102 mg of micronized eplerenone;
about 229 mg to about 234 mg of lactose;
about 43 mg to about 48 mg of microcrystalline cellulose; and
about 6 mg to about 10 mg of croscarmellose sodium.

The medicament composition just described optionally can additionally comprise:
about 0.5 mg to about 4 mg of sodium lauryl sulfate;
about 8 to about 12 mg of talc;
about 0.5 mg to about 3 mg of magnesium stearate; and
about 0.5 mg to about 4 mg of colloidal silicon dioxide.

The composition just described is preferably in the form of a capsule.

A portion of the contents of a capsule can be uncoated immediate-release cores such as those described above. A portion of the immediate-release cores can be coated with a rapidly disintegrating or dissolving coat for aesthetic, handling or stability purposes. Suitable materials for providing such a coat include polyvinylpyrrolidone, hydroxypropylcellulose, hydroxypropyl methylcellulose, polyethylene glycol, and polymethacrylates containing free amino groups. Such materials can further include plasticizers, antitack agents and/or diluents. An addition of about 3% of the weight of the core as coating material is generally regarded as providing a continuous coat for this size range. However, at least a portion of the contents of a capsule of the invention is a delayed-release formulation, for example immediate-release cores having an enteric coating as hereinabove described.

Controlled-Release Formulations

A composition of the present invention can include a controlled-release formulation of a second antihypertensive agent, including controlled-release formulations well known in the art, providing prolonged or sustained delivery of the drug—by various mechanisms. Such prolonged or sustained-release mechanisms can include, but are not limited to, pH sensitive release from the dosage form based on the changing pH of the small intestine; slow erosion of a tablet or capsule; retention in the stomach based on the physical properties of the formulation; bioadhesion of the dosage form to the mucosal lining of the intestinal tract; or enzymatic release of the second antihypertensive agent from the dosage form. The intended effect is to extend the time period over which the second antihypertensive agent is delivered to the site of action by manipulation of the dosage form. Thus, enteric-coated and enteric-coated controlled-release formulations are within the scope of the present invention.

Such controlled release formulations comprise the second antihypertensive agent in a desired antihypertensive amount as is well known. The controlled-release formulation may or may not be in a single dosage oral form that also contains the delayed-release formulation of the aldosterone antagonist.

Controlled-release dosage forms include extended-release dosage forms that permit at least a two-fold reduction in dosing frequency as compared to the drug presented as a conventional dosage form, and delayed-release dosage forms which release the drug at a time other than promptly after administration. The controlled-release formulation of the second antihypertensive agent can be, and preferably is, a sustained release formulation.

One type of controlled-release formulation, for example, is a matrix tablet formulation. Suitable matrix-forming materials are waxes (e.g., carnauba, bees wax, paraffin wax, ceresine, shellac wax, fatty acids and fatty alcohols); oils, hardened oils and fats (e.g., hardened rapeseed oil, castor oil, beef tallow, palm oil and soya bean oil); polymers (e.g., hydroxypropylcellulose, polyvinylpyrrolidone, hydroxypropyl methylcellulose, polyethylene glycol, methacrylates and carbomer); alginates; xanthan gums; and other carrier materials known to those of ordinary skill in the art to be useful as controlled-release matrix materials. Other suitable matrix tableting materials include, but are not limited to, microcrystalline cellulose, powdered cellulose and ethylcellulose. Other types of controlled-release compositions can achieve controlled release by use of granulates, coated powders, pellets, or the like, by use of multi-layering, and/or by use of suitable coatings. Standard coating procedures, such as those described, for example, in Remington's Pharmaceutical Sciences, 18th Edition (1990), can conveniently be used. Still other controlled-release compositions include an osmotic pump (such as described in British Patent No. 2 207 052), or combinations of the above.

The controlled release formulation of the second antihypertensive agent is combined into a single composition with the delayed-release formulation of the aldosterone antagonist, such that the amounts of the second antihypertensive agent and the aldosterone antagonist, e.g., eplerenone, in the composition provides the desired dosage.

In another embodiment, a composition of the invention includes micronized or nanoparticulate eplerenone in an immediate-release formulation in association with micronized or nanoparticulate eplerenone in a delayed-release formulation. The immediate-release formulation of eplerenone in such a composition can include an amount of eplerenone that is about 0.5% to about 50% of the total amount of eplerenone in the composition, with the delayed-release formulation containing the remainder of the micronized eplerenone. As a result, the final composition provides an amount of micronized or nanoparticulate eplerenone for immediate release following administration and an additional amount of micronized or nanoparticulate eplerenone for delayed release.

A typical coating composition for making a controlled-release component can contain an insoluble matrix polymer in an amount of about 15% to about 85% by weight of the coating composition, and a water-soluble material in an amount of about 15% to about 85% by weight of the coating composition. Optionally, an enteric polymer in an amount of about 0.1% to about 100% by weight of the coating composition may be used or included. Suitable insoluble matrix polymers include ethylcellulose, cellulose acetate butyrate, cellulose acetates, polymethacrylates containing quaternary ammonium groups, and other pharmaceutically acceptable polymers. Suitable water-soluble materials include polymers such as polyethylene glycol, hydroxypropyl-cellulose, hydroxypropyl methylcellulose, polyvinylpyrrolidone, polyvinyl alcohol; monomeric materials such as sugars (e.g., lactose, sucrose, fructose, mannitol and the like); salts (e.g., sodium chloride, potassium chloride and the like); organic acids (e.g., fumaric acid, succinic acid, lactic acid, tartaric acid and the like); and mixtures thereof. Suitable enteric polymers include hydroxypropyl methylcellulose acetate succinate, hydroxypropyl methylcellulose phthalate, polyvinyl acetate phthalate, cellulose acetate phthalate, cellulose acetate trimellitate, shellac, zein, polymethacrylates containing carboxyl groups and the like, as described above for delayed-release formulations.

The coating composition can be plasticized according to the properties of the coating blend such as the glass transition temperature of the main component or mixture of components or the solvent used for applying the coating compositions. Suitable plasticizers can be added from about 0.1% to about 50% by weight of the coating composition. Such plasticizers can be selected from, for example, the group consisting of diethyl phthalate, citrate esters, polyethylene glycol, glycerol, acetylated glycerides, acetylated citrate esters, dibutyl sebacate, castor oil and the like.

The coating composition can include a filler. The filler can comprise about 0.1% to about 100% by weight based on the total weight of the coating composition. The filler can be an insoluble material such as silicon dioxide, titanium dioxide, talc, kaolin, alumina, starch, powdered cellulose, microcrystalline cellulose, polacrilin potassium and the like.

The coating composition can be applied as a solution or latex in organic solvents, aqueous solvents or mixtures thereof. Where solutions are applied, the solvent is present in an amount of about 25% to about 99%, preferably about 85% to about 97%, by weight based on the total weight of solution. Suitable solvents are water, lower alcohols, lower chlorinated hydrocarbons, ketones and mixtures thereof. Where latexes are applied, the solvent is present in an amount of about 25% to about 97%, preferably about 60% to about 97%, by weight. The solvent can be predominantly water.

A typical matrix tablet can contain a diluent in an amount of about 15% to about 95% by weight. Additional diluents can be included in amounts from approximately 0.1% to about 65% by weight. These can be soluble materials such as lactose, mannitol, sorbitol and the like, or insoluble materials such as tribasic calcium phosphate, powdered cellulose or a starch (e.g., corn, wheat or potato starch).

Additionally, the tablets can contain a lubricant in an amount of about 0.1% to about 8% by weight. Lubricants can be selected from metal stearates, stearic acid, hydrogenated oils, such as soya bean oil or castor oil, sodium stearyl furnate, polytetrafluoroethylene, talc and the like.

The tablets can be enterically coated for aesthetic, handling or stability purposes, as well as to provide the desired delayed release of the drug. Suitable enteric coating materials are discussed above. The coating material can be added to any desired thickness but weight gains in the range about 15% to about 35% are typical, preferably about 20% to about 25%. The coat can be plasticized as also discussed above. The coating composition can include an antitack agent such as talc, kaolin, titanium dioxide, silicon dioxide, alumina, starch, polacrilin potassium, microcrystalline cellulose or the like.

Alternatively, the controlled-release component of a delayed-release tablet, when a second antihypertensive agent is present, can be provided in the form of controlled-release pellets containing the second antihypertensive agent, and the aldosterone antagonist included in the body of the enteric coated tablet. Such a tablet disintegrates after several hours in the intestine to release the aldosterone antagonist and the controlled-release pellets. In this embodiment, pellets can be present in an amount of about 1% to about 60%, preferably about 5% to about 50%, and more preferably about 5% to about 40%, by weight of the tablet. Suitable matrix materials for tablets of this type are microcrystalline cellulose, starches and the like.

A composition of the invention, where it is in a tablet or like form, can include two formulations as separate components, for example, in a multi-layer tablet, wherein one or more layers include the second antihypertensive agent, for example in a controlled-release form. Alternatively, the composition can be in the form of a tablet wherein an immediate release form of the second antihypertensive agent is present in the coating and the delayed-release formulation of the aldosterone antagonist constitutes the core.

The composition of the invention can be produced by providing a core containing the eplerenone formulation component coated with an enteric or delayed-release coating. The coated cores can then be compressed into tablets along with a powder mixture containing a second antihypertensive agent, or filled in combination with a second antihypertensive agent into a capsule shell. As a result, the final composition provides an amount of the second antihypertensive agent for immediate release following administration and an additional amount of eplerenone for delayed-release.

Additional controlled-release formulations can be prepared by appropriate modification of the formulations and methods disclosed in, for example, U.S. Pat. No. 5,190,765 to Jao et al., U.S. Pat. No. 5,160,744 to Jao et al., U.S. Pat. No. 5,082,668 to Wong et al., U.S. Pat. No. 4,847,093 to Ayer et al., European Patent Application No. 0 572 942, European Patent Application No. 0 284 039, European Patent Application No. 0 238 189, International Patent Publication No. WO 94/27582, International Patent Publication No. WO 92/13547, and International Patent Publication No. WO 92/00729.

Methods of Treatment

The present invention also is directed to a method of treating or preventing a condition or disorder where therapy or prophylaxis with an aldosterone antagonist is indicated, the method comprising orally administering one or more of the pharmaceutical compositions described herein to a mammal such as a human patient. The dosage regimen to prevent, give relief from, or ameliorate the condition or disorder preferably corresponds to a once-a-day oral dosage, and more preferably to the 10 mg, 20 mg, 50 mg or 100 mg eplerenone oral unit dosages discussed above, but can be modified in accordance with a variety of factors. These factors include the type, age, weight, sex, diet, and medical condition of the patient and the severity of the disease, condition or disorder. Thus, the dosage regimen actually employed can vary widely and therefore deviate from the preferred dosage regimen set forth above.

Initial treatment of a patient suffering from a condition or disorder where treatment with an aldosterone antagonist is indicated can begin with the dosages indicated above. Treatment is generally continued as necessary over a period of several weeks to several months or years until the condition or disorder has been controlled or eliminated. Patients undergoing treatment with the compositions disclosed herein can be routinely monitored by any of the methods well known in the art to determine the effectiveness of therapy. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of aldosterone antagonist exhibiting satisfactory effectiveness is administered, and so that administration is continued only so long as is necessary to successfully treat the condition or disorder.

Method for Preparation of Formulation

The present invention also is directed to methods for preparation of delayed-release compositions comprising micronized or nanoparticulate eplerenone or other aldosterone antagonist. Where tablets or capsules are desired, methods such as wet granulation, dry granulation or direct compression or encapsulation methods can be employed. This discussion will center on preparation of formulations containing the preferred aldosterone antagonist, eplerenone. It is to be understood that similar techniques can be used for preparing a formulation containing a second antihypertensive agent, where desired, or that preparations from the patent or other literature relating to such compounds can be used, as is well known.

Wet granulation is a preferred method of preparing tablets. In a wet granulation process, micronized or nanoparticulate eplerenone (and, if desired, any of the carrier materials) is initially milled or micronized to the desired particle size using a conventional mill or grinder. Such milling or grinding techniques are well known in the art, as are methods for ascertaining the resulting particle size and distribution.

The milled or micronized eplerenone is then blended, for example in a high shear mixer granulator, planetary mixer, a twin-shell blender or sigma mixer, with one or more of the carrier materials. Typically, the drug is blended with the diluent(s), disintegrant(s), binding agent(s) and, optionally, wetting agent(s) in this step although all or a portion of one or more of the carrier materials can be added in a later step.

For example, where microcrystalline cellulose is employed as a diluent, addition of a portion of the microcrystalline cellulose during this blending step and addition of the remaining portion after the drying step discussed below typically increases the hardness and/or decreases the friability of the tablets produced. In this situation, preferably about 40% to about 50% of the microcrystalline cellulose is added intragranularly and about 50% to about 60% of the microcrystalline cellulose is added extragranularly. In addition, this step of the process preferably comprises the blending of eplerenone, lactose, microcrystalline cellulose, hydroxypropyl methylcellulose and, optionally, sodium lauryl sulfate. Blending times as short as three minutes can provide a dry powder mixture having a sufficiently uniform distribution of eplerenone.

Water is then added to the dry powder mixture and the mixture is blended for an additional period of time. The water can be added to the mixture at once, gradually over a period of time, or in several portions over a period of time. The water preferably is added gradually over a period of time, preferably at least about 3 to about 5 minutes. An additional period of mixing, generally at least about 1 to about 3 minutes, after the water addition is complete helps to ensure uniform distribution of the water in the mixture and results in a suitable wet granulated mixture.

It is generally preferred that the wet granulated mixture comprise about 25% to about 45% water by weight. Although a higher or lower water content can be acceptable for certain formulations, a lower water content generally reduces the effectiveness of the wet granulation step in producing granules having the desired compressibility and flowability properties, whereas a higher water content generally causes an increase in granule size.

The wet granulated mixture is then dried, for example, in an oven or a fluidized bed dryer, preferably a fluidized bed drier. If desired, the wet granulated mixture can be wet milled, extruded or spheronized prior to drying, although wet milling is preferred. For the drying process, conditions such as inlet air temperature and drying time are adjusted to achieve the desired moisture content for the dried mixture. Increasing moisture content from about 2% to about 4% can decrease initial tablet hardness.

To the extent necessary, the dry granules are then reduced in size in preparation for compression. Conventional particle size reduction equipment such as oscillators or Fitz mills can be employed.

The dry granules are then placed in a suitable blender such as a twin-shell blender and the lubricant, anti-adherent agent and any additional carrier materials are added. Although blending times depend in part upon the process equipment used, it has been discovered that blending times of at least about 5 to 25 minutes are generally preferred. In a preferred embodiment of this step of the invention, talc and the remaining portion of microcrystalline cellulose are added to the granules and the mixture blended for an additional period of time, preferably a period of time sufficient to achieve a blend uniformity characterized by a relative standard deviation value of about 6% or less.

Magnesium stearate is then added to the mixture and the mixture is blended for an additional period of time. As noted above, where the diluents include microcrystalline cellulose, the addition of a portion of the microcrystalline cellulose during this step can increase tablet hardness. In addition, increasing the amount of magnesium stearate can decrease tablet hardness and increase friability and disintegration time.

This blended mixture is then compressed into tablets (or encapsulated if capsules are to be prepared) to the desired weight and hardness using appropriate size tooling. Conventional compression and encapsulation techniques known to those of ordinary skill in the art can be employed. Coating techniques as hereinabove described or known to those of ordinary skill in the art are then employed to provide a delayed-release formulation.

### EXAMPLES

The following examples illustrate aspects of the present invention but should not be construed as limitations. The experimental procedures used to generate the data shown are discussed in more detail below. The symbols and conventions used in these examples are consistent with those used in the contemporary pharmaceutical literature. Unless otherwise stated, (i) all percentages recited in these examples are weight percents based on total composition weight, (ii) total composition weight for capsules is the total capsule fill weight and does not include the weight of the actual capsule employed, and (iii) enteric coated tablets are coated as discussed hereinabove.

### Example 1

25 mg Dose Tablet

A 25 mg dose immediate release tablet (tablet diameter of 7/32″) is prepared having the following composition:

TABLE 1

| Ingredient | % by weight | Amount (mg) |
|---|---|---|
| eplerenone | 29.41 | 25.00 |
| lactose monohydrate (#310, NF) | 42.00 | 35.70 |
| microcrystalline cellulose | 18.09 (7.50% | 15.38 |
| (NF, Avicel ® PH101) | intragranular | |
| | plus 10.59% | |
| | extragranular) | |
| croscarmellose sodium | 5.00 | 4.25 |
| (NF, Ac-Di-Sol ™) | | |
| hydroxypropyl methylcellulose | 3.00 | 2.55 |
| (#2910, USP, Pharmacoat ™ 603) | | |
| sodium lauryl sulfate (NF) | 1.00 | 0.85 |
| talc (USP) | 1.00 | 0.85 |
| magnesium stearate (NF) | 0.50 | 0.42 |
| Total | 100 | 85 |

The lactose monohydrate used in each of the examples of the application is commercially available from Formost Farms, Baraboo, Wis. The Avicel® brand of microcrystalline cellulose and the Ac-Di-Sol™ brand of croscarmellose sodium are used in each of the examples of the application. Both compounds are commercially available from FMC Corporation, Chicago, Ill. The Pharmacoat™ 603 brand of hydroxypropyl methylcellulose is used in each of the examples of the application. This compound is commercially available from Shin-Etsu Chemical Co. Ltd. The sodium lauryl sulfate used in each of the examples of the application is commercially available from Henkel Corporation, Cincinnati, Ohio. The talc used in each of the examples of the application is commercially available from Cyprus Foote Mineral Co., Kings Mountain, N.C., or Luzenac America, Inc., Englewood, Colo. The magnesium stearate used in each of the examples of the application is commercially available from Mallinckrodt Inc., St. Louis, Mo.

Tablets so prepared are coated with an enteric coating to provide delayed-release of eplerenone. To form the coating solution, Eudragit™ RS (11.4 kg), Eudragit™ RL (0.81 kg), triethyl citrate (1.20 kg) and sodium lauryl sulfate (0.275 kg) are dissolved in ethanol (89.81 kg). Talc (6.82 kg) is then added to the solution.

The coating layer is applied to the tablets using a Wurster bottom spray coater until an actual coat weight of about 25-30% based on total tablet weight is obtained. The coated tablets are dried for about 30 minutes at a temperature of about 40° C., then cooled to ambient temperature.

### Example 2

50 mg Dose Tablet

A 50 mg dose tablet (tablet diameter of 9/32″) is prepared having the following composition:

TABLE 2

| Ingredient | % by weight | Amount (mg) |
|---|---|---|
| eplerenone | 29.41 | 50.00 |
| lactose monohydrate (#310, NF) | 42.00 | 71.40 |
| microcrystalline cellulose | 18.09 (7.50% | 30.75 |
| (NF, Avicel ® PH101) | intragranular | |
| | plus 10.59% | |
| | extragranular) | |
| croscarmellose sodium | 5.00 | 8.50 |
| (NF, Ac-Di-Sol ™) | | |
| hydroxypropyl methylcellulose | 3.00 | 5.10 |
| (#2910, USP, Pharmacoat ™ 603) | | |
| sodium lauryl sulfate (NF) | 1.00 | 1.70 |
| talc (USP) | 1.00 | 1.70 |
| magnesium stearate (NF) | 0.50 | 0.85 |
| Total | 100 | 170 |

These tablets are coated as discussed in Example 1 to provide a delayed-release formulation.

### Example 3

100 mg Dose Tablets

A 100 mg dose tablet formulation (tablet diameter of 12/32″) is prepared having the following composition:

TABLE 3

| Ingredient | % by weight | Amount (mg) |
|---|---|---|
| eplerenone | 29.41 | 100.00 |
| lactose monohydrate (#310, NF) | 42.00 | 142.80 |
| microcrystalline cellulose | 18.09 (7.50% | 61.50 |
| (NF, Avicel ® PH101) | intragranular | |
| | plus 10.59% | |
| | extragranular) | |
| croscarmellose sodium | 5.00 | 17.00 |
| (NF, Ac-Di-Sol ™) | | |
| hydroxypropyl methylcellulose | 3.00 | 10.20 |
| (#2910, USP, Pharmacoat ™ 603) | | |
| sodium lauryl sulfate (NF) | 1.00 | 3.40 |
| talc (USP) | 1.00 | 3.40 |
| magnesium stearate (NF) | 0.50 | 1.70 |
| Total | 100 | 340 |

These tablets are coated as discussed in Example 1 to provide a delayed-release formulation.

### Example 4

10 mg Dose Capsules

A 10 mg dose capsule formulation is prepared having the following composition:

TABLE 4

| | Amount | Representative |
|---|---|---|
| Ingredient | (mg) | batch amount (kg) |
| eplerenone | 10.0 | 1.00 |
| lactose, hydrous NF | 306.8 | 30.68 |
| microcrystalline cellulose, NF | 60.0 | 6.00 |
| talc, USP | 10.0 | 1.00 |
| croscarmellose sodium, NF | 8.0 | 0.80 |
| sodium lauryl sulfate, NF | 2.0 | 0.20 |
| colloidal silicon dioxide, NF | 2.0 | 0.20 |
| magnesium stearate, NF | 1.2 | 0.12 |
| Total capsule fill weight | 400.0 | 40.00 |
| Hard gelatin capsule, Size #0, | 1 capsule | 100,000 capsules |
| White Opaque | | |

A coating formulation is prepared by dispersing 3.3 parts magnesium stearate in 31 parts water containing 0.1 part simethicone emulsion USP and adding it to 65.6 parts Eudragit™ NE30D. The coating formulation is applied to 3500 gram portions of the eplerenone-containing beads by the Wurster process using a spray rate of 40 grams per minute and an inlet air temperature of 40° C. to provide an enteric coating constituting about 20-25% of the final formulation weight, excluding the weight of the capsule.

### Example 5

25 mg Dose Capsules

A 25 mg dose capsule formulation is prepared having the following composition:

TABLE 5

| | | Representative |
|---|---|---|
| | Amount | batch amount |
| Ingredient | (mg) | (kg) |
| eplerenone | 25.0 | 2.50 |
| lactose, hydrous NF | 294.1 | 29.41 |
| microcrystalline cellulose, NF | 57.7 | 5.77 |
| talc, USP | 10.0 | 1.00 |
| croscarmellose sodium, NF | 8.0 | 0.80 |
| sodium lauryl sulfate, NF | 2.0 | 0.20 |
| colloidal silicon dioxide, NF | 2.0 | 0.20 |
| magnesium stearate, NF | 1.2 | 0.12 |
| Total capsule fill weight | 400.0 | 40.00 |
| Hard gelatin capsule, Size #0, | 1 capsule | 100,000 capsules |
| White Opaque | | |

The eplerenone-containing core particles so formed are coated as discussed in Example 4 prior to filling into the capsules.

### Example 6

50 mg Dose Capsules

A 50 mg dose capsule formulation is prepared having the following composition:

TABLE 6

| | | Representative |
|---|---|---|
| | Amount | batch amount |
| Ingredient | (mg) | (kg) |
| eplerenone | 50.0 | 5.00 |
| lactose, hydrous NF | 273.2 | 27.32 |
| microcrystalline cellulose, NF | 53.6 | 5.36 |
| talc, USP | 10.0 | 1.00 |
| croscarmellose sodium, NF | 8.0 | 0.80 |
| sodium lauryl sulfate, NF | 2.0 | 0.20 |
| colloidal silicon dioxide, NF | 2.0 | 0.20 |
| magnesium stearate, NF | 1.2 | 0.12 |
| Total capsule fill weight | 400.0 | 40.00 |
| Hard gelatin capsule, Size #0, | 1 capsule | 100,000 capsules |
| White Opaque | | |

The eplerenone-containing core particles so formed are coated as discussed in Example 4 prior to filling into the capsules.

### Example 7

100 mg Dose Capsules

A 100 mg dose capsule formulation is prepared having the following composition:

TABLE 7

| | | Representative |
|---|---|---|
| | Amount | batch amount |
| Ingredient | (mg) | (kg) |
| eplerenone | 100.0 | 10.00 |
| lactose, hydrous NF | 231.4 | 23.14 |
| microcrystalline cellulose, NF | 45.4 | 4.54 |
| talc, USP | 10.0 | 1.00 |
| croscarmellose sodium, NF | 8.0 | 0.80 |
| sodium lauryl sulfate, NF | 2.0 | 0.20 |
| colloidal silicon dioxide, NF | 2.0 | 0.20 |
| magnesium stearate, NF | 1.2 | 0.12 |
| Total capsule fill weight | 400.0 | 40.00 |
| Hard gelatin capsule, Size #0, | 1 capsule | 100,000 capsules |
| White Opaque | | |

The eplerenone-containing core particles so formed are coated as discussed in Example 4 prior to filling into the capsules.

### Example 8

Preparation of Tablets

The ingredients of the pharmaceutical compositions of the present invention can be prepared in accordance with acceptable pharmaceutical manufacturing for small scale preparations.

An illustrative formulation process using the starting materials of Table 8 is set forth below. The process can be operated as a single batch reaction or as two or more parallel batch reactions.

TABLE 8

| | | Representative |
|---|---|---|
| | % by | batch amount |
| Ingredient | weight | (kg) |
| eplerenone | 29.41 | 4.412 |
| lactose monohydrate (#310, NF) | 42.00 | 6.3 |
| microcrystalline cellulose | 7.50 | 1.125 |
| (intragranular) (NF, Avicel ® PH101) | | |
| croscarmellose sodium | 5.00 | 0.75 |
| (NF, Ac-Di-Sol ™) | | |
| hydroxypropyl methylcellulose | 3.00 | 0.45 |
| (#2910, USP, Pharmacoat ™ 603) | | |
| sodium lauryl sulfate (NF) | 1.00 | 0.15 |
| talc (USP) | 1.00 | 0.15 |
| microcrystalline cellulose | 10.59 | 1.588 |
| (intragranular) (NF, Avicel ® PH101) | | |
| magnesium stearate (NF) | 0.50 | 0.075 |
| Total | 100.00 | 15.00 |

Milling: The eplerenone is milled in a jet mill. The resulting milled eplerenone has D₁₀, D₅₀ and D₉₀ values of 2.65 μm, 23.3 μm and 99.93 μm, respectively. In other words, 10%, 50% and 90% of the eplerenone particles were less than 2.65 μm, 23.3 μm and 99.93 μm, respectively, in size. A pin mill is preferred for preparation on a manufacturing scale.

Dry mixing: A 65 L Niro™ Fielder granulator is loaded with the lactose, eplerenone, Avicel®, Ac-Di-Sol™, Pharmacoat™ 603 and sodium lauryl sulfate in this order. These materials are mixed to homogeneity (about 3 minutes) with the main blade on the slow main blade setting and the chopper blade on the slow chopper blade setting. For manufacturing scale, a machine such as a Bukler Perkins™ 1000L granulator can be used.

Wet granulation: The dry powder mixture is wet granulated using USP water. The main blade and chopper blade of the granulator are placed on the fast speed setting. Water in an amount of 5 kg is added to the mixture over a period of about 3 minutes using a Masterflex™ water pump, model 7524-00 (24″ tubing). The rate of water addition is about 1.66 kg/minute. The wet mixture is blended for an additional minute to ensure uniform distribution of the water in the granulation. The wet granulated mixture is about 38% water by weight.

Drying: The wet granulation is placed in a Freund™ Flo-coater (FLF-15) fluid bed dryer. The inlet air temperature is adjusted to about 68° C. and the granulation is dried in the fluid bed dryer to reduce the moisture content to 0.5% to 2.5%. Moisture content is monitored using a Computrac™ Moisture Analyzer.

Dry screening: The dry granules were passed through a Fitz mill with a 20 mesh screen, knives forward, and 2400 rpm speed.

Blending and lubrication: The dry granules are then placed in a Paterson-Kelley 2 cubic foot V-blender. The talc and extragranular Avicel® 101 are placed on top of the granules and the mixture blended to homogeneity (about 10 minutes). The magnesium stearate is placed on top of the mixture and the mixture blended for an additional three minutes. A Croff™ flow blender can be used for large scale preparations.

Compression: The granules are then compressed on a Killian™ tablet press to the desired weight and hardness using appropriate sized tooling. The target weight, size and hardness for 25, 50 and 100 mg tablets is as set forth in Table 8A below:

TABLE 8A

| Eplerenone | Tablet | Tooling size (inch) | Target hardness |
|---|---|---|---|
| dosage (mg) | weight (mg) | (round, standard concave) | range (kP) |
| 25 | 85 | 7/32 | 3-9 |
| 50 | 170 | 9/32 | 5-14 |
| 100 | 340 | 12/32 | 8-16 |

Film coating: When coated with a water-soluble or dispersible coating, the above tablets are suitable as an immediate release formulation. Coating as discussed in Examples 1 or 4 provides a delayed-release formulation.

As various changes can be made in the above formulations and methods without departing from the scope of the invention, it is intended that all matter contained in the above description be interpreted as illustrative and not in a limiting sense. All patent documents listed herein are incorporated by reference.

1. A pharmaceutical composition for administration to a subject mammal exhibiting a diurnal cycle of plasma aldosterone concentration having an acrophase, the composition comprising a therapeutically effective amount of a delayed-release formulation of an aldosterone antagonist drug which, when orally administered about 6 to about 12 hours prior to the acrophase, provides a profile of plasma drug concentration corresponding substantially to the diurnal cycle of plasma aldosterone concentration.

2. The composition of claim 1 wherein the profile of plasma drug concentration corresponds to the diurnal cycle of plasma aldosterone concentration substantially as depicted in FIG. 1.

3. A pharmaceutical composition comprising a delayed-release formulation of an aldosterone antagonist drug in a therapeutically effective amount, the composition exhibiting a release profile, as determined by a suitable test, in which:
(a) zero to about 20% by weight of the drug is released from the formulation at about 4 hours after initiation of the test; and
(b) about 50% to 100% by weight of the drug is released from the formulation within a time period of about 3 hours beginning at a time about 4 to about 12 hours after initiation of the test.

4. The composition of claim 3 wherein the test is conducted according to U.S. Pharmacopeia 24, Test No. 711, using apparatus 2 at 50 rpm, with an aqueous dissolution medium containing 1% sodium dodecyl sulfate at 37° C., and wherein release is measured by dissolution of the drug in the medium.

5. The composition of claim 3 wherein zero to about 10% by weight of the drug is released from the formulation at about 4 hours after initiation of the test.

6. The composition of claim 3 wherein zero to about 20% by weight of the drug is released from the formulation at about 6 hours after initiation of the test.

7. The composition of claim 3 wherein zero to about 10% by weight of the drug is released from the formulation at about 6 hours after initiation of the test.

8. The composition of claim 3 wherein about 70% to 100% by weight of the drug is released from the formulation within said time period of about 3 hours.

9. The composition of claim 1 wherein that further comprises a second formulation comprising a therapeutically effective amount of a second antihypertensive agent.

10. The composition of claim 9 wherein said second antihypertensive agent is selected from a diuretic, a sympatholytic agent, an ACE inhibitor, a vasopeptidase, a calcium channel blocker, a direct vasodilator, a renin inhibitor, and an angiotensin II antagonist.

11. The composition of claim 9 wherein the second formulation containing the second antihypertensive agent exhibits a release profile that is different from the release profile exhibited by the delayed-release formulation containing the aldosterone antagonist.

12. The composition of claim 11 wherein the second formulation is an immediate-release formulation.

13. The composition of claim 11 wherein the second formulation is an extended-release formulation.

14. The composition of claim 1 wherein the aldosterone antagonist is eplerenone.

15. The composition of claim 1 that is in the form of an enteric coated tablet.

16. The composition of claim 15 wherein the tablet comprises a core comprising an immediate-release formulation of the aldosterone antagonist substantially enclosed within an enteric coating.

17. The composition of claim 1 that is in the form of a capsule containing enteric coated pellets.

18. The composition of claim 17 wherein the pellets each comprise a core comprising an immediate-release formulation of the aldosterone antagonist substantially enclosed within an enteric coating.

19. A method of treating a mammal exhibiting (a) circadian rhythm in aldosterone secretion having an acrophase and (b) an aldosterone-mediated disease or disorder, the method comprising orally administering to the mammal a composition of any of claims 1-18 about 6 to about 12 hours prior to the acrophase.

20. The method of claim 19 wherein the mammal is a human.

21. The method of claim 20 wherein the disease or disorder is elevated blood pressure.

22. The method of claim 20 wherein the acrophase occurs at the end of a sleep period and the composition is orally administered prior to the sleep period.
